(19) United States
(12) Patent Application Publication
(10) Pub. No.: US 2025/0314657 A1
(43) Pub. Date: Oct. 9, 2025

(54) METHODS FOR DETECTION OF ANALYTES

(71) Applicant: Quantum-Si Incorporated, Branford, CT (US)

(72) Inventors: Brian Reed, Madison, CT (US); Marco Ribezzi-Crivellari, paris (FR); Sebastian Hutchinson, Branford, CT (US)

(73) Assignee: Quantum-Si Incorporated, Branford, CT (US)

(21) Appl. No.: 19/173,718

(22) Filed: Apr. 8, 2025

Related U.S. Application Data
(60) Provisional application No. 63/631,167, filed on Apr. 8, 2024.

(51) Int. Cl. G01N 33/58; G01N 33/68

(52) U.S. Cl. CPC G01N 33/582; G01N 33/6818; G01N 2440/14

(57) ABSTRACT

The disclosure provides methods and compositions that enable the characterization of analytes in a sample, including the identification of polypeptides having one or more post-translational modifications. In some embodiments, the disclosure provides methods of determining a concentration of an analyte in a sample based at least in part on a count of detected series of signal pulses. In some embodiments, the disclosure provides methods of determining one or more chemical characteristics of an analyte (e.g., a polypeptide). In some embodiments, the disclosure provides a method (e.g., a single-molecule method) comprising contacting a single polypeptide with one or more post-translational modification-specific (PTM-specific) affinity reagents; and identifying whether the single polypeptide comprises a post-translational modification (PTM) by determining a luminescence signature representative of the binding interaction(s) between the single polypeptide and the one or more PTM-specific affinity reagents.

### CROSS-REFERENCE TO RELATED APPLICATIONS

This application claims priority under 35 U.S.C. § 119 (e) to U.S. Provisional Patent Application No. 63/631,167, filed Apr. 8, 2024, which is hereby incorporated by reference in its entirety.

### BACKGROUND

Proteins represent the fundamental building blocks of life, driving key biological and cellular processes. Protein function is driven by its structure, including its sequence. In adjacent fields, like genomics, advances in sequencing technology have proven extremely valuable in improving our understanding of the progression of complex human disease.

Post-translational modifications (e.g., phosphorylation) impact protein function and structure. Identification of polypeptides having post-translational modifications and determining the specific location of a post-translational modification within a polypeptide has historically been challenging, as methodologies have been generally limited to ensemble-based methods.

### SUMMARY

In some aspects, the disclosure provides methods of sample analysis. In some embodiments, a method of sample analysis comprises contacting a capture reagent with a sample comprising one or more analytes, where the capture reagent binds an analyte of the sample to form a first complex; contacting the first complex with a composition comprising one or more affinity reagents and one or more secondary reporters, where at least one affinity reagent binds the analyte of the first complex; detecting at least one series of signal pulses, where each series of signal pulses is indicative of a series of binding events between the one or more secondary reporters and an affinity reagent bound to the analyte of the first complex; and determining a concentration of the analyte in the sample based at least in part on a count of detected series of signal pulses.

In some embodiments, the at least one series of signal pulses comprises: a first set of at least one series of signal pulses indicative of a first series of binding events between one or more secondary reporters and a first affinity reagent bound to the analyte, and a second set of at least one series of signal pulses indicative of a second series of binding events between one or more secondary reporters and a second affinity reagent bound to the analyte. In some embodiments, the first affinity reagent is different from the second affinity reagent. In some embodiments, the first affinity reagent binds to a first site on the analyte, and the second affinity reagent binds to a second site on the analyte. In some embodiments, the first site does not comprise a post-translational modification (PTM), and the second site comprises a PTM. In some embodiments, the first site comprises a first PTM, and the second site comprises a second PTM.

In some embodiments, the capture reagent is attached to a surface of a substrate. In some embodiments, the method further comprises, prior to contacting the capture reagent with the sample: contacting a substrate with the capture reagent, where a surface of the substrate comprises an attachment moiety that forms a covalent or non-covalent attachment between the capture reagent and the surface.

In some embodiments, the capture moiety is attached within a first compartment of an array comprising a plurality of compartments. In some embodiments, the concentration of the analyte in the sample is determined based at least in part on the count of the detected series of signal pulses of the first compartment. In some embodiments, the method comprises detecting at least one series of signal pulses in each of at least two compartments of the array. In some embodiments, the concentration of the analyte in the sample is determined based at least in part on the count of the detected series of signal pulses of the at least two compartments.

In some embodiments, a second compartment of the array comprises a second capture moiety bound to a second analyte that is different from the analyte bound by the capture moiety in the first compartment. In some embodiments, the method further comprises: detecting at least one series of signal pulses, where each series of signal pulses is indicative of a series of binding events between the one or more secondary reporters and an affinity reagent bound to the second analyte in the second compartment.

In some embodiments, the method further comprises: determining a concentration of the second analyte in the sample based at least in part on a count of detected series of signal pulses of the second compartment. In some embodiments, the method further comprises: determining a concentration of the first and second analytes in the sample based on one or more characteristics of the detected series of signal pulses of the first and second compartments. In some embodiments, the concentration is a relative concentration of the first analyte to the second analyte. In some embodiments, the concentration is an absolute concentration of each of the first and second analytes. In some embodiments, the concentration is the relative concentration is determined based at least in part on a ratio of the count of detected series of signal pulses of the first compartment to the count of detected series of signal pulses of the second compartment.

In some embodiments, the method further comprises: determining a relative concentration of the first and second analytes in the sample based at least in part on a ratio of a total count of detected series of signal pulses across two or more compartments each comprising the first analyte relative to a total count of detected series of signal pulses across two or more compartments each comprising the second analyte.

In some embodiments, at least one series of signal pulses detected in each of the first and second compartments comprises a series of signal pulses indicative of analyte binding by an affinity reagent of the same type. In some embodiments, the series of signal pulses detected in each of the first and second compartments indicate that the analytes in the first and second chambers are encoded by a single gene. In some embodiments, at least one series of signal pulses detected in the first compartment comprises a series of signal pulses indicative of analyte binding by an affinity reagent of a different type from the at least one series of signal pulses detected in the second compartment. In some embodiments, the series of signal pulses detected in each of the first and second compartments indicate that the analytes in the first and second chambers are different isoforms encoded by a single gene.

In some embodiments, the method further comprises distinguishing signal pulses indicative of the series of binding events from signal pulses resulting from noise based at least in part on a characteristic pattern in a detected series of signal pulses. In some embodiments, the method further comprises removing the signal pulses resulting from noise prior to determining the concentration of the analyte in the sample. In some embodiments, the characteristic pattern comprises a pulse duration and/or an interpulse duration of the detected series of signal pulses. In some embodiments, the pulse duration comprises an average duration of pulses of the detected series of signal pulses. In some embodiments, the interpulse duration comprises an average duration between pulses of the detected series of signal pulses.

In some embodiments, the capture reagent is contacted with a single composition comprising the sample, the one or more affinity reagents, and the one or more secondary reporters. In some embodiments, the capture reagent is conjugated to a barcode. In some embodiments, the barcode is a peptide barcode. In some embodiments, the method further comprises: removing the barcode from the capture reagent; and determining a sequence of the barcode, where the sequence of the barcode is indicative of the analyte to which the capture reagent binds. In some embodiments, the capture moiety is attached to a surface through a linkage group comprising the barcode.

In some embodiments, the at least one affinity reagent binds the analyte of the first complex to form a second complex comprising the analyte, the capture reagent, and an affinity reagent. In some embodiments, the capture reagent of the second complex is attached to a surface through a first linkage group, and the affinity reagent is attached to the surface through a second linkage group. In some embodiments, the capture reagent comprises a first oligonucleotide, and the first oligonucleotide is hybridized to a first surface-immobilized oligonucleotide to form the first linkage group. In some embodiments, the affinity reagent comprises a second oligonucleotide, and the second oligonucleotide is hybridized to a second surface-immobilized oligonucleotide to form the second linkage group. In some embodiments, the method further comprises, prior to detecting the at least one series of signal pulses: forming the second complex; and contacting the second complex with the surface to form the first and second linkage groups.

In some aspects, the disclosure provides methods of sample analysis. In some embodiments, a method of sample analysis comprises contacting a capture reagent with a sample comprising one or more analytes, where the capture reagent binds an analyte of the sample to form a first complex; contacting the first complex with one or more affinity reagents; detecting at least one series of signal pulses, where each series of signal pulses is indicative of a series of binding events between the one or more affinity reagents and the analyte; and determining a concentration of the analyte in the sample based at least in part on a count of detected series of signal pulses.

In some embodiments, the at least one series of signal pulses comprises: a first set of at least one series of signal pulses indicative of a first series of binding events between a first affinity reagent and the analyte, and a second set of at least one series of signal pulses indicative of a second series of binding events between a second affinity reagent and the analyte. In some embodiments, the first affinity reagent is different from the second affinity reagent. In some embodiments, the first affinity reagent binds to a first site on the analyte, and the second affinity reagent binds to a second site on the analyte. In some embodiments, the first site does not comprise a post-translational modification (PTM), and the second site comprises a PTM. In some embodiments, the first site comprises a first PTM, and the second site comprises a second PTM.

In some embodiments, the capture reagent is attached to a surface of a substrate. In some embodiments, the method further comprises, prior to contacting the capture reagent with the sample: contacting a substrate with the capture reagent, where a surface of the substrate comprises an attachment moiety that forms a covalent or non-covalent attachment between the capture reagent and the surface.

In some embodiments, the capture moiety is attached within a first compartment of an array comprising a plurality of compartments. In some embodiments, the concentration of the analyte in the sample is determined based at least in part on the count of the detected series of signal pulses of the first compartment. In some embodiments, the method comprises detecting at least one series of signal pulses in each of at least two compartments of the array. In some embodiments, the concentration of the analyte in the sample is determined based at least in part on the count of the detected series of signal pulses of the at least two compartments.

In some embodiments, a second compartment of the array comprises a second capture moiety bound to a second analyte that is different from the analyte bound by the capture moiety in the first compartment. In some embodiments, the method further comprises: detecting at least one series of signal pulses, where each series of signal pulses is indicative of a series of binding events between the one or more affinity reagents and the second analyte in the second compartment.

In some embodiments, the method further comprises: determining a concentration of the second analyte in the sample based at least in part on a count of detected series of signal pulses of the second compartment. In some embodiments, the method further comprises: determining a concentration of the first and second analytes in the sample based on one or more characteristics of the detected series of signal pulses of the first and second compartments. In some embodiments, the concentration is a relative concentration of the first analyte to the second analyte. In some embodiments, the concentration is an absolute concentration of each of the first and second analytes. In some embodiments, the relative concentration is determined based at least in part on a ratio of the count of detected series of signal pulses of the first compartment to the count of detected series of signal pulses of the second compartment.

In some embodiments, the method further comprises: determining a relative concentration of the first and second analytes in the sample based at least in part on a ratio of a total count of detected series of signal pulses across two or more compartments each comprising the first analyte relative to a total count of detected series of signal pulses across two or more compartments each comprising the second analyte.

In some embodiments, at least one series of signal pulses detected in each of the first and second compartments comprises a series of signal pulses indicative of analyte binding by an affinity reagent of the same type. In some embodiments, the series of signal pulses detected in each of the first and second compartments indicate that the analytes in the first and second chambers are encoded by a single gene. In some embodiments, at least one series of signal pulses detected in the first compartment comprises a series of signal pulses indicative of analyte binding by an affinity reagent of a different type from the at least one series of signal pulses detected in the second compartment. In some embodiments, the series of signal pulses detected in each of the first and second compartments indicate that the analytes in the first and second chambers are different isoforms encoded by a single gene.

In some embodiments, the method further comprises distinguishing signal pulses indicative of the series of binding events from signal pulses resulting from noise based at least in part on a characteristic pattern in a detected series of signal pulses. In some embodiments, the method further comprises removing the signal pulses resulting from noise prior to determining the concentration of the analyte in the sample. In some embodiments, the characteristic pattern comprises a pulse duration and/or an interpulse duration of the detected series of signal pulses. In some embodiments, the pulse duration comprises an average duration of pulses of the detected series of signal pulses. In some embodiments, the interpulse duration comprises an average duration between pulses of the detected series of signal pulses.

In some embodiments, the capture reagent is contacted with a single composition comprising the sample and the one or more affinity reagents. In some embodiments, the capture reagent is conjugated to a barcode. In some embodiments, the barcode is a peptide barcode. In some embodiments, the method further comprises: removing the barcode from the capture reagent; and determining a sequence of the barcode, where the sequence of the barcode is indicative of the analyte to which the capture reagent binds. In some embodiments, the capture moiety is attached to a surface through a linkage group comprising the barcode.

In some embodiments, at least one affinity reagent binds the analyte of the first complex to form a second complex comprising the analyte, the capture reagent, and an affinity reagent. In some embodiments, the capture reagent of the second complex is attached to a surface through a first linkage group, and the affinity reagent is attached to the surface through a second linkage group. In some embodiments, the capture reagent comprises a first oligonucleotide, and the first oligonucleotide is hybridized to a first surface-immobilized oligonucleotide to form the first linkage group. In some embodiments, the affinity reagent comprises a second oligonucleotide, and the second oligonucleotide is hybridized to a second surface-immobilized oligonucleotide to form the second linkage group. In some embodiments, the method further comprises, prior to detecting the at least one series of signal pulses: forming the second complex; and contacting the second complex with the surface to form the first and second linkage groups.

In some embodiments, the first complex is contacted with a composition comprising the one or more affinity reagents and one or more secondary reporters. In some embodiments, the method further comprises: detecting at least one series of signal pulses indicative of a series of binding events between the one or more secondary reporters and an affinity reagent bound to the analyte of the first complex.

In some aspects, the disclosure provides methods of determining one or more chemical characteristics of a polypeptide. In some embodiments, a method of determining one or more chemical characteristics of a polypeptide comprises contacting a polypeptide with a composition comprising one or more affinity reagents and one or more secondary reporters, where at least one affinity reagent binds the polypeptide; detecting at least one series of signal pulses indicative of a series of binding events between the one or more secondary reporters and an affinity reagent bound to the polypeptide; and determining one or more chemical characteristics of the polypeptide based on one or more characteristics of the at least one series of signal pulses.

In some embodiments, each series of signal pulses is indicative of a single binding event between an affinity reagent and the polypeptide. In some embodiments, each series of signal pulses is indicative of a duration in which the polypeptide is bound by affinity reagent. In some embodiments, each series of signal pulses is separated from another by a duration in which the polypeptide is unbound by affinity reagent.

In some embodiments, the polypeptide is attached to a surface. In some embodiments, the polypeptide is attached to the surface through a capture reagent that binds the polypeptide. In some embodiments, the capture reagent binds to a site on the polypeptide that is different from a site to which the at least one affinity reagent binds. In some embodiments, the capture reagent comprises an antibody, an antigen-binding portion of an antibody (e.g., a single-chain antibody variable fragment (scFv) or V_{H}H fragment), or an aptamer.

In some embodiments, the method further comprises contacting the polypeptide with a capture reagent that binds the polypeptide, where the capture reagent is attached to a surface. In some embodiments, the polypeptide is contacted with the capture reagent prior to contacting the polypeptide with the composition. In some embodiments, the polypeptide is contacted with the capture reagent in a single composition comprising the polypeptide, the one or more affinity reagents, and the one or more secondary reporters. In some embodiments, the method further comprises, prior to contacting the polypeptide with the capture reagent: contacting the capture reagent with the surface, where the surface comprises an attachment moiety that forms a covalent or non-covalent attachment between the capture reagent and the surface. In some embodiments, the attachment moiety comprises an avidin protein, and where the capture reagent comprises a biotin moiety that is bound by the avidin protein.

In some embodiments, contacting the polypeptide with the composition comprises: contacting the polypeptide with a single composition comprising the one or more affinity reagents and the one or more secondary reporters. In some embodiments, contacting the polypeptide with the composition comprises: contacting the polypeptide with a first composition comprising the one or more affinity reagents and a second composition comprising the one or more secondary reporters.

In some embodiments, the one or more secondary reporters bind the affinity reagent at a faster rate than a time required for the affinity reagent to dissociate from the polypeptide. In some embodiments, the one or more affinity reagents comprise one or more antibodies, antigen-binding portions of an antibody (e.g., a single-chain antibody variable fragment (scFv) or V_{H}H fragment), or aptamers.

In some embodiments, the one or more secondary reporters comprise one or more terminal amino acid recognizers. In some embodiments, each of the one or more affinity reagents is conjugated to a tag peptide. In some embodiments, each of the one or more secondary reporters binds the tag peptide of an affinity reagent. In some embodiments, each series of signal pulses is indicative of a series of binding events between the one or more secondary reporters and the tag peptide of an affinity reagent bound to the polypeptide. In some embodiments, each of the one or more secondary reporters comprises a terminal amino acid recognizer that binds a terminal amino acid of the tag peptide.

In some embodiments, each of the one or more affinity reagents is conjugated to a tag oligonucleotide. In some embodiments, each of the one or more secondary reporters comprises a complementary oligonucleotide that hybridizes to the tag oligonucleotide of an affinity reagent. In some embodiments, each series of signal pulses is indicative of a series of hybridization events between the one or more secondary reporters and the tag oligonucleotide of an affinity reagent bound to the polypeptide.

In some embodiments, each of the one or more secondary reporters comprises a luminescent label. In some embodiments, the polypeptide is a full-length protein or a polypeptide fragment thereof.

In some embodiments, the one or more affinity reagents comprise at least two affinity reagents that bind different proteoforms of the polypeptide.

In some embodiments, the one or more affinity reagents comprise at least two affinity reagents that bind different epitopes of a single proteoform of the polypeptide. In some embodiments, the one or more affinity reagents comprise a first affinity reagent that binds a first epitope of the polypeptide and a second affinity reagent that binds a second epitope of the polypeptide. In some embodiments, the first epitope does not comprise a post-translational modification (PTM), and where the second epitope comprises a PTM. In some embodiments, the first affinity reagent is a protein-specific affinity reagent that binds different proteoforms of the polypeptide, and where the second affinity reagent is a PTM-specific affinity reagent that binds a specific proteoform of the polypeptide.

In some embodiments, the one or more characteristics of the at least one series of signal pulses comprise a first recognition segment duration of a first series of signal pulses. In some embodiments, the first recognition segment duration comprises a length of time during which the first series of signal pulses is detected. In some embodiments, the first recognition segment duration is characteristic of a dissociation rate of affinity reagent binding and/or a dissociation rate of capture reagent binding. In some embodiments, the one or more characteristics of the at least one series of signal pulses comprise an average of two or more recognition segment durations.

In some embodiments, the one or more characteristics of the at least one series of signal pulses comprise an intersegment duration between two recognition segment durations. In some embodiments, the intersegment duration comprises a length of time between two successively detected series of signal pulses. In some embodiments, the intersegment duration is characteristic of an association rate of affinity reagent binding and/or an association rate of capture reagent binding. In some embodiments, the one or more characteristics of the at least one series of signal pulses comprise an average of two or more intersegment durations.

In some embodiments, the one or more characteristics of the at least one series of signal pulses comprise a first pulse duration of a first series of signal pulses. In some embodiments, the first pulse duration comprises an average duration of pulses of the first series of signal pulses. In some embodiments, the first pulse duration is characteristic of a dissociation rate of secondary reporter binding.

In some embodiments, the one or more characteristics of the at least one series of signal pulses comprise a first interpulse duration of a first series of signal pulses. In some embodiments, the first interpulse duration comprises an average duration between pulses of the first series of signal pulses. In some embodiments, the first interpulse duration is characteristic of an association rate of secondary reporter binding.

In some embodiments, determining the one or more chemical characteristics of the polypeptide comprises identifying the polypeptide. In some embodiments, determining the one or more chemical characteristics of the polypeptide comprises identifying one or more post-translational modifications of the polypeptide. In some embodiments, determining the one or more chemical characteristics of the polypeptide comprises determining a concentration of the polypeptide in a sample from which it was derived.

In some aspects, the disclosure relates to methods of proteoform analysis. For example, the inventors of the disclosure have identified a novel methodology for the identification of post-translational modifications (PTMs) within one or more polypeptides. Specifically, the inventors have identified a methodology for identifying PTMs in a single molecule context (and not merely in an ensemble context). These methods enable the determination of precise locations of PTMs within a single polypeptide and single-molecule level determinations of proteoform distributions within a sample.

Accordingly, in some aspects, the disclosure provides a single-molecule method comprising (a) contacting a single polypeptide with one or more post-translational modification-specific (PTM-specific) affinity reagents; and (b) identifying whether the single polypeptide comprises a post-translational modification (PTM) by determining a luminescence signature representative of the binding interaction(s) between the single polypeptide and the one or more PTM-specific affinity reagents.

In some embodiments, the method further comprises (c) contacting the single polypeptide with one or more terminal amino acid recognition molecules; and (d) detecting a series of signal pulses indicative of association of the one or more terminal amino acid recognition molecules with successive amino acids exposed at a terminus of the single polypeptide while the single polypeptide is being degraded.

Further aspects of the disclosure provide a method of polypeptide sequencing comprising: (a) contacting an array (e.g., a chip array) comprising a plurality of compartments with a plurality of polypeptides; (b) immobilizing each polypeptide of the plurality of polypeptides to a surface of the array (e.g., chip array); (c) contacting the plurality of polypeptides with one or more post-translational modification-specific (PTM-specific) affinity reagents; and (d) identifying whether each polypeptide comprises a post-translational modification (PTM) by determining the luminescence signature representative of the binding interaction(s) between each polypeptide and the one or more PTM-specific affinity reagents.

In some embodiments, the method further comprises: (c) contacting each polypeptide with one or more terminal amino acid recognition molecules; and (f) detecting a series of signal pulses indicative of association of the one or more terminal amino acid recognition molecules with successive amino acids exposed at a terminus of each polypeptide while each polypeptide is being degraded, thereby sequencing each polypeptide.

Further aspects of the disclosure provide a method of characterizing proteoforms of a polypeptide comprising: (a) contacting an array (e.g., chip array) comprising a plurality of compartments with a sample comprising a first proteoform of a polypeptide and a second proteoform of a polypeptide, wherein the post-translational modification (PTM) profile of the first proteoform is different than the PTM profile of the second proteoform; (b) immobilizing the first proteoform to a surface of a first compartment of the array (e.g., chip array) and the second proteoform to a surface of a second compartment of the array (e.g., chip array); (c) contacting the first proteoform and the second proteoform with one or more post-translational modification-specific (PTM-specific) affinity reagents; and (d) identifying whether the first proteoform and/or the second proteoform comprises a post-translational modification (PTM) by determining a luminescence signature representative of the binding interaction(s) between each proteoform and the one or more PTM-specific affinity reagents.

In some embodiments, the method further comprises: (e) contacting the first proteoform and/or the second proteoform with one or more terminal amino acid recognition molecules; and (f) detecting a series of signal pulses indicative of association of the one or more terminal amino acid recognition molecules with successive amino acids exposed at a terminus of each proteoform while each proteoform is being degraded, thereby sequencing the first proteoform and the second proteoform.

In some embodiments, the one or more PTM-specific affinity reagents are antibodies or aptamers. In some embodiments, the one or more PTM-specific affinity reagents specifically bind to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination.

In some embodiments, the one or more PTM-specific affinity reagents specifically bind to phospho-tyrosine, phospho-serine, or phospho-threonine.

In some embodiments, the one or more PTM-specific affinity reagents is labeled. In some embodiments, the label is a luminescent label or a conductivity label. In some embodiments, the luminescent label comprises at least one fluorophore dye molecule.

In some embodiments, the luminescent label comprises 20 or fewer fluorophore dye molecules.

In some embodiments, the polypeptide(s) are contacted with two or more PTM-specific affinity reagents at the same time. In some embodiments, each of the two or more PTM-specific affinity reagents comprise a unique label relative to the other PTM-specific affinity reagents.

In some embodiments, the polypeptide(s) are contacted in series with a first PTM-specific affinity reagent and a second PTM-specific affinity reagent, optionally wherein the first PTM-specific affinity reagent is removed (e.g., by washing) prior to addition of the second PTM-specific affinity reagent.

In some embodiments, determining the luminescence signature comprises detecting a series of signal pulses indicative of association of the one or more PTM-specific affinity reagents with the PTM of the polypeptide(s).

In some embodiments, detecting a series of signal pulses indicative of association of the one or more PTM-specific affinity reagents with the PTM of the polypeptide(s) allows for a determination of the type of amino acids located at positions in proximity to the PTM of the polypeptide(s).

In some embodiments, detecting a series of signal pulses indicative of association of the one or more PTM-specific affinity reagents with the PTM of the polypeptide(s) allows for a determination of the location of the PTM within the polypeptide(s).

In some embodiments, detecting a series of signal pulses indicative of association of the one or more PTM-specific affinity reagents with the PTM of the polypeptide(s) assists with a determination of the amino acid sequence of the polypeptide(s).

In some embodiments, the PTM is to an amino acid comprising a phosphorylation, a glycosylation, acctylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination. In some embodiments, the PTM is phospho-tyrosine, phospho-serine, or phospho-threonine.

In some embodiments, contacting the polypeptide(s) with one or more terminal amino acid recognition molecules further comprises contacting the polypeptide(s) with a cleaving reagent. In some embodiments, the cleaving reagent is an aminopeptidase.

In some embodiments, the method allows for identification of the presence of the PTM at any location in the polypeptide(s).

In some embodiments, the method further comprises washing the polypeptide(s) after determining the luminescence signature of the polypeptide(s) in the presence of the one or more PTM-specific affinity reagents.

In some embodiments, the method further comprises fragmenting the polypeptide(s) prior to step (a). In some embodiments, the fragmenting is done by cleaving (e.g., chemically cleaving) and/or digesting (e.g., enzymatically digesting using a peptidase) the polypeptide(s).

In some embodiments, association of the one or more terminal amino acid recognition molecules with each type of amino acid exposed at the terminus produces a characteristic pattern in the series of signal pulses that is different from other types of amino acids exposed at the terminus, optionally wherein the characteristic pattern comprises a portion of the series of signal pulses.

In some embodiments, a signal pulse of the characteristic pattern corresponds to an individual association event between a terminal amino acid recognition molecule and an amino acid exposed at the terminus. In some embodiments, the signal pulse of the characteristic pattern comprises a pulse duration that is characteristic of a dissociation rate of binding between the terminal amino acid recognition molecule and the amino acid exposed at the terminus. In some embodiments, each signal pulse of the characteristic pattern is separated from another by an interpulse duration that is characteristic of an association rate of terminal amino acid recognition molecule binding.

In some embodiments, the characteristic pattern corresponds to a series of reversible terminal amino acid recognition molecule binding interactions with the amino acid exposed at the terminus of the single polypeptide molecule.

In some embodiments, the characteristic pattern is indicative of the amino acid exposed at the terminus of the single polypeptide molecule and an amino acid at a contiguous position.

Further aspects of the disclosure provide a composition comprising three or more PTM-specific affinity reagents. In some embodiments, the three or more PTM-specific affinity reagents are antibodies or aptamers. In some embodiments, the three or more PTM-specific affinity reagents specifically bind to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination.

In some embodiments, the composition comprises a PTM-specific affinity reagent that specifically binds to phospho-tyrosine, a PTM-specific affinity reagent that specifically binds to phospho-serine, and a PTM-specific affinity reagent that specifically binds to phospho-threonine.

In some embodiments, the one or more PTM-specific affinity reagents are labeled. In some embodiments, the label is a luminescent label or a conductivity label. In some embodiments, the luminescent label comprises at least one fluorophore dye molecule. In some embodiments, the luminescent label comprises 20 or fewer fluorophore dye molecules. In some embodiments, each of the three or more PTM-specific affinity reagents comprise a unique label relative to the other PTM-specific affinity reagents.

In some embodiments, the composition further comprises a sample comprising polypeptides. In some embodiments, the composition further comprises a cleaving reagent.

The details of certain embodiments of the disclosure are set forth in the Detailed Description. Other features, objects, and advantages of the disclosure will be apparent from the Examples, Drawings, and Claims.

### BRIEF DESCRIPTION OF THE DRAWINGS

The accompanying Drawings, which constitute a part of this specification, illustrate several embodiments of the disclosure and together with the accompanying description, serve to explain the principles of the disclosure.

FIGS. 1A-1B provide data showing the ability of an exemplary method of the disclosure to identify that the presence and location of a phospho-tyrosine (a post-translational modification) in a model polypeptide.

FIG. 2 provides data showing the ability of an exemplary method of the disclosure to determine that a full-length polypeptide comprises a post-translational modification.

FIG. 3 demonstrates that the methods of the disclosure can be performed on a chip (e.g., by immobilizing polypeptide targets to surfaces of the chip) and in solution (e.g., by contacting the polypeptide target(s) with PTM-specific affinity reagents (e.g., PTM-specific nanobodies) in solution.

FIGS. 4A-4C provide schematics of an example single-molecule method for identification and characterization of the pattern of post-translational modifications present on a single polypeptide.

FIG. 5 provides an example method in which an affinity reagent is capable of binding a site on a polypeptide of post-translational modification independently of the modification state of the site.

FIG. 6 provides an example method that utilizes dye cycling. A polypeptide of interest is immobilized to a surface of a chip and contacted with an affinity reagent and a luminescently labeled secondary reporter.

FIG. 7 provides an example multiplexed method that utilizes dye cycling. A polypeptide of interest is immobilized to a surface of a chip and contacted with two affinity reagents and two luminescently labeled secondary reporters having different luminescent properties.

FIGS. 8A-8B show example methods that utilize dye cycling. In this approach, an affinity reagent is conjugated to a tag peptide designed to be recognized by an N-terminal amino acid recognizer. Peptide-labeled affinity reagents are then used to detect analytes (e.g., proteins), which can be immobilized directly to a surface (FIG. 8A) or through a surface-immobilized capture reagent (FIG. 8B) that binds the analyte.

FIGS. 9A-9H show representative experimental results demonstrating dye cycling in model systems. FIG. 9A shows the kinetic signature of an anti-Lysozyme VHH bound to Lysozyme in a single aperture as reported by the recognition of the N-terminal phenylalanine by PS610. As shown by the shaded regions, interaction between the anti-lysozyme and lysozyme target protein creates a distinct ROI^{ON}. FIG. 9B is a plot showing exponential decay of the duration of the specific ROI^{ON}. FIG. 9C shows results demonstrating recognition of GFP and Lysozyme in a single run with GFP: Lysozyme in a 1:1 ratio in mixture with peptide-labeled affinity reagents (anti-GFP R-Lag16 VHH, anti-Lysozyme VHH F-1ZV5) and recognizers (PS1220 (R), PS610 (F)). FIG. 9D shows representative kinetic signatures of R-Lag16 (bottom two panels) and F-1ZV5 (top two panels) in independent apertures containing either GFP or lysozyme immobilized. FIG. 9E generally depicts an assay in which differently labeled affinity reagents were combined and evaluated in a mixture with lysozyme and recognizers. FIG. 9F shows a representative kinetic signature of the differently labeled affinity reagents binding lysozyme in a single aperture. In the context of a mixture of affinity reagents, the binding events between single VHH molecules to the Lysozyme were observed independently, as reported by different recognizers (PS1223 for VHH having N-terminal L; PS610 for VHH having N-terminal F; and PS1220 for VHH having N-terminal R). FIG. 9G generally depicts an assay in which GFP labeled with peptide having either N-terminal F, L, or R amino acids were evaluated in mixture with an immobilized anti-GFP. FIG. 9H shows representative kinetic signatures of anti-GFP binding the differently labeled GFP molecules.

FIGS. 10A-10D show representative experimental results of an immuno-sandwich assay utilizing the dye cycling approach. FIG. 10A shows a scheme representing the dynamic sandwich assay for lysozyme quantification with a capture reagent (1ZV5 VHH), an affinity reagent labeled with N-terminal R for detection (R-1ZVH VHH), and a secondary affinity reagent reporting the sandwich (PS1220 recognizer). FIG. 10B shows representative kinetic signatures obtained in mixtures having 1 ng/ml (top panel) or 1 pg/mL (bottom panel) lysozyme. FIG. 10C shows 2-dimensional histograms of measured ROIs in mixtures having 10 ng/mL lysozyme (top panel), 100 μg/mL lysozyme (middle panel), or no lysozyme as control (bottom panel). FIG. 10D is a titration curve for lysozyme showing response in number of ROI per concentration of analyte and concentration of Lysozyme in pg/mL.

FIGS. 11A-11D show representative experimental results of GFP labeled with a N-terminal arginine peptide (R-GFP) evaluated with Lag16 as capture antibody and PS1220. FIG. 11A depicts a general scheme of this approach and illustrates the relation between number of detection events (ROI^{ON}; shaded regions) and analyte binding events to the capture antibody. FIG. 11B is a titration curve for R-GFP as analyte showing response in number of ROI per concentration of analyte and concentration of GFP in pg/mL. FIGS. 11C-11D show comparative results from these studies using either Lag16 or Lag9 as capture reagent.

FIGS. 12A-12D show representative experimental results evaluating the use of antibody capture reagents. FIG. 12A depicts a general scheme of IL-6 analyte bound by IgG capture reagent and recognized by peptide-labeled affinity reagent. FIGS. 12B-12C show example results from runs carried out at different IL-6 concentrations. FIG. 12D shows example results from spike-in titration experiments in serum with IL-6 (left plot), lysozyme (middle plot), or GFP (right plot) as analyte.

FIGS. 13A-13B show anti-Tau IgGs that were utilized as affinity and/or capture reagents in studies with Tau as analyte.

FIGS. 14A-14C show representative experimental results in which the Tau isoform 2N4R was immobilized directly on chip in mixture with Tau-12 antibody (25 nM), which carried a peptide label having N-terminal arginine, and the arginine recognizer PS1220.

FIGS. 15A-15B show representative experimental results in which the 2N4R and 0N3R Tau isoforms were immobilized directly on chip in mixture with the 2N4R-specific antibody, Tau-2N, which carried a peptide label having N-terminal arginine, and the arginine recognizer PS1220.

FIGS. 16A-16C show representative experimental results in which the 2N4R and 0N3R Tau isoforms were immobilized directly on chip in a mixture to permit total and isoform-specific Tau recognition: F-labeled anti-2N IgG (2N4R isoform-specific), R-labeled HT7 (total Tau), PS610 (F recognizer), and PS1220 (R recognizer).

FIGS. 17A-17E show representative experimental results in which the Tau antibody HT7 was immobilized to chip surface to serve as capture reagent for binding either the 2N4R or 1N3R isoform, each of which was evaluated in mixture with Tau-12-R, anti-2N-F, PS1220 (R recognizer), and PS610 (F recognizer).

FIGS. 18A-18B show representative experimental results demonstrating recognition of phosphorylated Tau peptide.

FIGS. 19A-19B show an example of multiplexing based on barcoding the capture reagent in accordance with methods described herein.

FIG. 20 shows an example of conditional capture in which analyte is captured on surface after forming a complex with affinity reagent and capture reagent in accordance with methods described herein.

### DETAILED DESCRIPTION

Aspects of the disclosure relate to compositions and methods for identification of post-translation modifications (PTMs) in one or more polypeptides.

Compositions and methods for obtaining luminescence signatures and collecting data relating to binding interactions between an affinity reagent (e.g., a labeled reagent) and a polypeptide, including compositions and methods for polypeptide sequencing, are described more fully in PCT International Publication No. WO2020102741A1, filed Nov. 15, 2019, PCT International Publication No. WO2021236983A2, filed May 20, 2021, PCT International Publication No. WO2023122769A2, filed Dec. 22, 2022, PCT International Publication No. WO2024031031A2, filed Aug. 3, 2023, and PCT International Publication No. WO2024086832A1, filed Oct. 20, 2023, each of which is incorporated by reference in its entirety.

In some aspects, the disclosure provided methods of determining one or more chemical characteristics of an analyte (e.g., one or more analytes in a sample). In some embodiments, the analyte comprises a polypeptide, such as a protein (e.g., a full-length protein or a peptide fragment thereof). In some embodiments, the methods described herein can be used to identify or characterize proteoforms of a polypeptide. As used herein and known in the art, the term “proteoforms” refers to different molecular forms (e.g., isoforms) of a protein product encoded by a single gene. The proteoform of a polypeptide encompasses the translated amino acid sequence of the polypeptide and post-translational modifications of the polypeptide. In reference to a particular polypeptide, different proteoforms refer to the range of different structures of a protein product arising from a single gene. As the presence or concentration of a particular proteoform may increase or decrease in abnormal physiological states, protein characterization at the proteoform level has a crucial importance to fully understand biological processes. Accordingly, in some embodiments, the methods of the disclosure can be used for characterizing analyte isoforms in a biological sample, which can in turn provide meaningful information for therapeutic and diagnostic purposes.

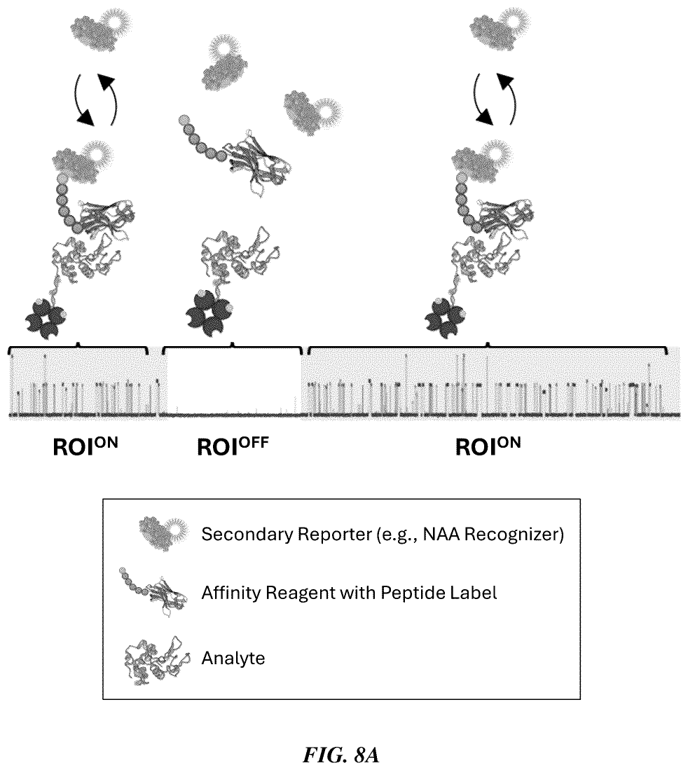

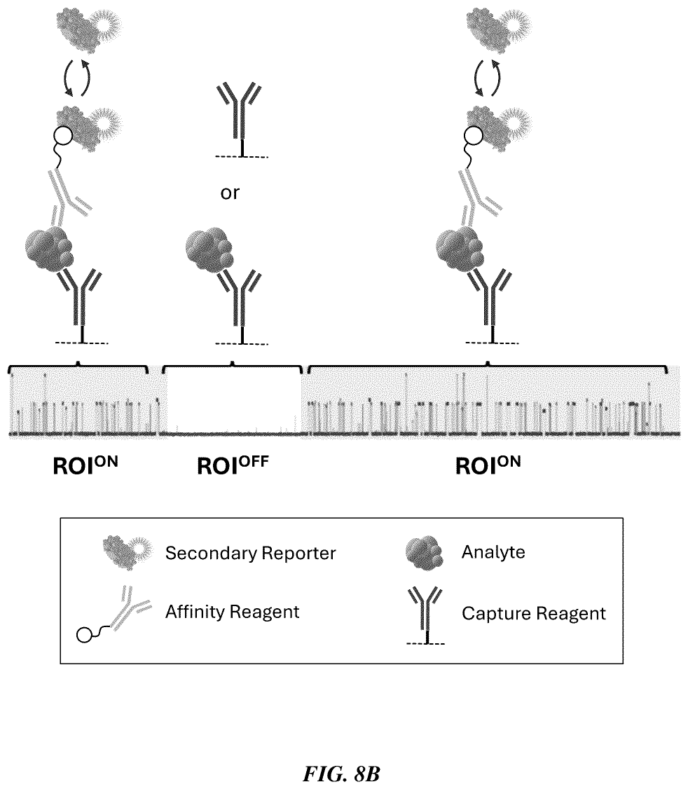

For example, in some aspects, the disclosure provides methods of sample analysis that comprise detecting at least one series of signal pulses, where each series of signal pulses is indicative of a series of binding events between one or more secondary reporters and an affinity reagent bound to an analyte of a sample. As generally illustrated in FIGS. 8A-8B, each series of signal pulses corresponds to a recognition segment duration (“ROI^{ON}”), and the inventors have demonstrated that the number of detected recognition segment durations in a signal is indicative of analyte concentration in a sample. Thus, in some embodiments, the methods described herein can be used to determine the amount of an analyte or relative amounts of more than one analyte in a sample, which can be used for therapeutic and/or diagnostic purposes.

In some embodiments, the methods described herein comprise determining one or more characteristics of one or more analytes in a sample. In some embodiments, the sample is a biological sample (e.g., a cell, serum, blood, or tissue sample). In some embodiments, the sample is derived from a biological source (e.g., a cell, serum, blood, or tissue sample). In some embodiments, the sample is or is derived from a biological fluid (e.g., blood, serum, urine, saliva, cerebrospinal fluid). In some embodiments, the one or more analytes comprise one or more polypeptides. The term “polypeptide” as used herein can refer to a polymeric form of amino acids of any length (e.g., at least 10, at least 20, at least 30, at least 50, at least 100, 5-500, 20-500, 100-500, 5-50, 10-100, 250-500, 500 or more amino acids in length).

In some embodiments, a polypeptide refers to a protein (e.g., a full-length protein). In some embodiments, a protein can refer to any full-length, natively folded protein, such as a naturally occurring protein that has not been artificially fragmented (e.g., digested) into smaller peptide fragments. In some embodiments, a protein comprises a polymeric form of amino acids of at least 50 amino acids in length (e.g., at least 75, at least 100, at least 150, at least 250, 50-500, 50-250, 50-100, 100-250, 200-400, 250-500, 500 or more amino acids in length). In some embodiments, a protein has a molecular weight of at least 5 kilodaltons (e.g., at least 10, at least 15, at least 25, at least 50, 10-100, 10-50, 25-100, 25-50, 50-250, or 50-100 kilodaltons in size).

In some embodiments, a polypeptide refers to a peptide, such as a peptide fragment of a full-length protein. In some embodiments, a peptide refers to a polymeric form of amino acids of any length that is shorter than the full-length protein from which it is derived. In some embodiments, a peptide comprises a polymeric form of amino acids of at least 5 amino acids in length (e.g., at least 10, at least 15, at least 20, at least 25, 5-50, 10-50, 15-40, 20-60, 20-40, or 15-60 amino acids in length). Accordingly, in some embodiments, a polypeptide can refer to a peptide fragment of a protein, and the methods described herein can further comprise fragmenting a protein to produce the peptide and one or more peptide fragments of the protein. In some embodiments, the fragmenting comprises cleaving (e.g., chemically cleaving) and/or digesting (e.g., enzymatically digesting using a peptidase, such as trypsin or proteinase K) the protein to produce the peptide fragments thereof.

In some embodiments, the methods described herein comprise identifying and/or characterizing proteoforms of a polypeptide. In some embodiments, different proteoforms of a polypeptide comprise different post-translational modifications (PTMs) that occur, typically catalyzed by enzymes, after translation of the protein. A PTM generally refers to the covalent addition of a functional group to a protein, proteolytic cleavage of a protein, or degradation of one or more regions of a protein. Examples of PTMs are known in the art and include, without limitation, phosphorylation, glycosylation, acctylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, and ubiquitination.

In some embodiments, the methods described herein comprise determining the presence, location, and/or abundance of one or more PTMs in a polypeptide based at least in part on a detected series of signal pulses. Suitable techniques for obtaining such signal pulse information and determining characteristic patterns therein have been described more fully, for example, in PCT International Publication Nos. WO2020102741A1, WO2021236983A2, WO2023122769A2, WO2024031031A2, and WO2024086832A1, each of which is incorporated by reference in its entirety.

### Dye Cycling

In some embodiments of the disclosure, for methods having slow binding kinetics, photobleaching or photo damage (e.g., photobleaching of a fluorophore or photo damage to a polypeptide of interest, e.g., an immobilized polypeptide) can be a rate-limiting factor in identifying and/or sequencing, e.g., of a polypeptide of interest. Accordingly, it is desirable in some instances to mitigate photobleaching. The inventors of the present disclosure have identified a strategy referred to as “dye cycling,” that utilizes a luminescently labeled secondary reporter to provide a separation between the polypeptide being identified and/or sequenced (e.g., a polypeptide of interest) and the luminescent label or dye. Affinity reagents (i.e., affinity reagents such as antibodies that bind to the polypeptide being identified and/or sequenced (e.g., a polypeptide of interest) are not directly labeled with a luminescent label or dye but rather carry a specific ‘tag’ that is detected by one or more luminescently (e.g., fluorescently) labeled secondary reporters. In some embodiments, the relatively slow (and longer) binding events between the polypeptide of interest and the affinity reagent are detected as a series of short pulses, a “recognition segment,” as the durations of these recognition segments and their spacing can be used to detect the kinetics of the affinity regents. Slow binding kinetics of an affinity reagent can increase the likelihood of photobleaching occurring before sufficient binding data is received. Slow binding kinetics can also result in fewer binding events per experiment, increasing the difficulty of detection. Using secondary reporters mitigates these challenges.

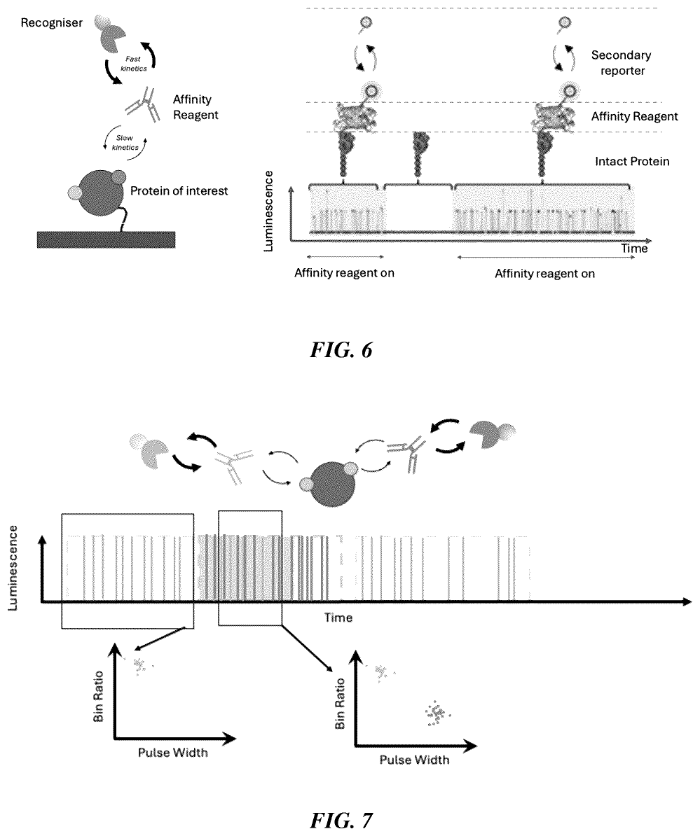

As demonstrated in FIG. 6 (an example schematic of a dye cycling methodology), such methods involve a primary affinity reagent that binds to a polypeptide/protein of interest (slow kinetics) and a luminescently labeled recognizer (secondary reporter) that binds to the affinity reagent. The binding of the affinity reagent to the polypeptide of interest can be detected because of the more rapid binding interaction between the affinity reagent and the recognizer. When the affinity reagent is not bound to the polypeptide of interest, then there will be no signal pulses detected as the recognizer cannot directly interact with the polypeptide of interest.

FIGS. 8A-8B provide further example schematics illustrating dye cycling methodologies of the disclosure. As shown, in some embodiments, the methods comprise detecting at least one series of signal pulses, where each series of signal pulses (denoted as “ROI^{ON}” or alternatively referred to herein as a “recognition segment” or “recognition segment duration”) is indicative of a series of binding events between one or more secondary reporters and an affinity reagent bound to an analyte. In some embodiments, each series of signal pulses is separated from another by a duration in which the analyte is unbound by affinity reagent (denoted as “ROI^{OFF}” or alternatively referred to herein as an “intersegment duration”).

In some embodiments, an affinity reagent is configured to bind an analyte, and a secondary reporter is configured to bind the affinity reagent. In some embodiments, the secondary reporter binds the affinity reagent at a faster rate than a time required for the affinity reagent to dissociate from the analyte, which permits detection of signal pulses while the affinity reagent is bound to the analyte. Thus, in some embodiments, each series of signal pulses is indicative of a single binding event between an affinity reagent and the analyte, and each signal pulse of a series is indicative of a single binding event between a secondary reporter and the affinity reagent.

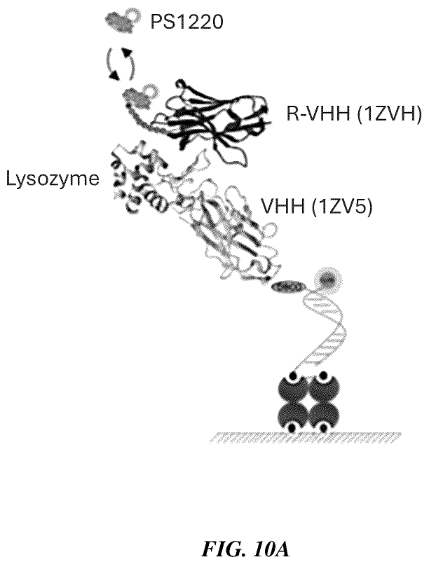

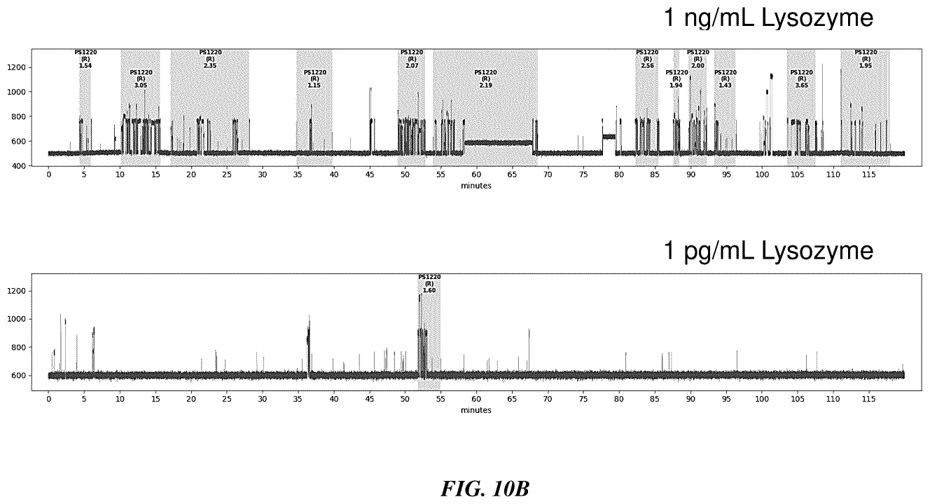

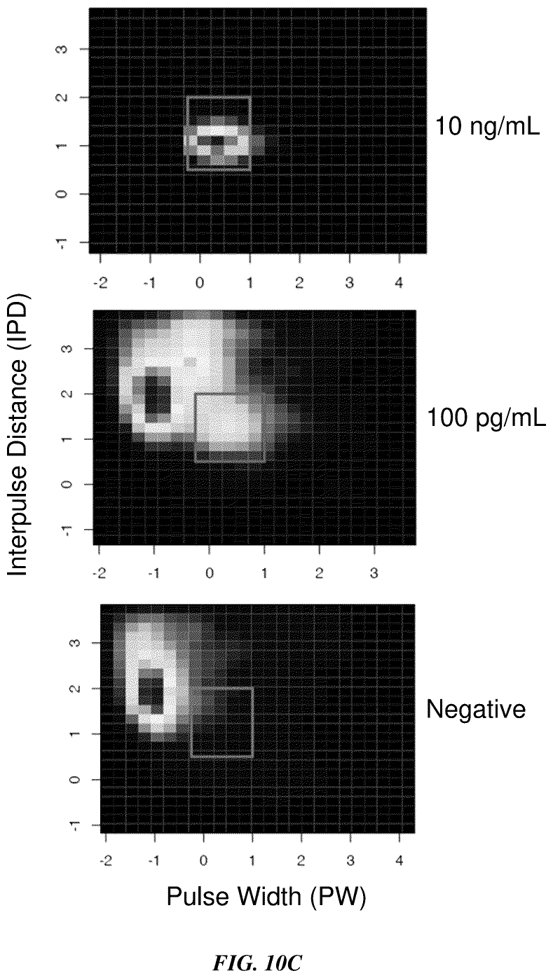

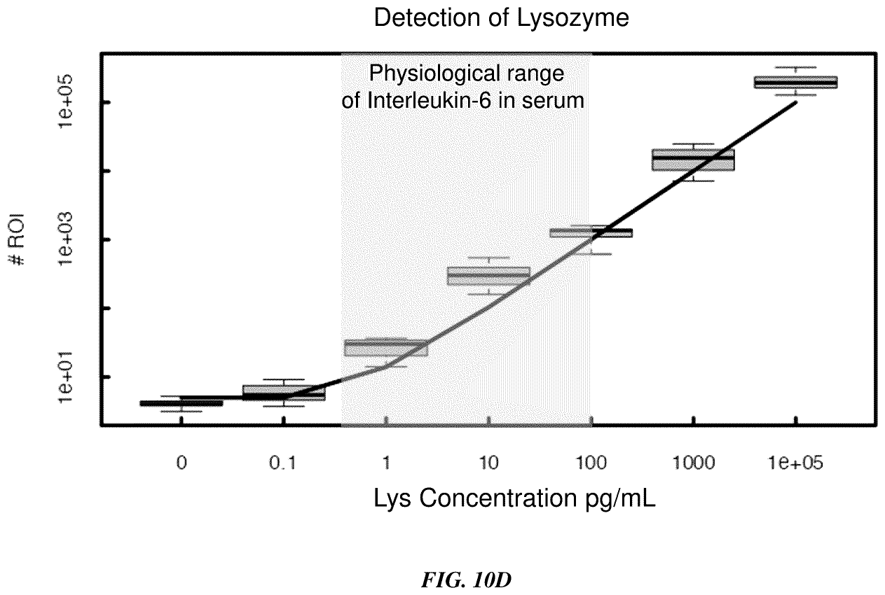

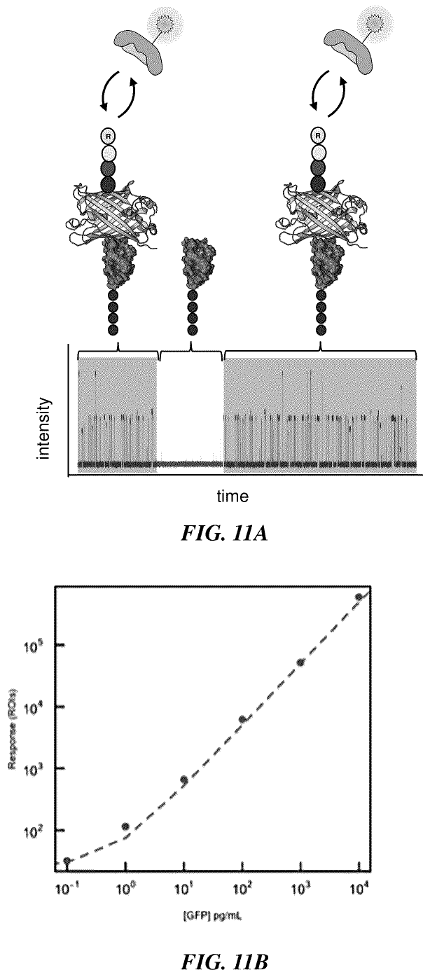

In some embodiments, the detected series of signal pulses can be used to determine one or more characteristics of an analyte (e.g., a polypeptide) in a sample. For example, in some embodiments, the methods described herein comprise determining a concentration of an analyte in a sample based at least in part on a count of detected series of signal pulses (e.g., a number of recognition segments or “ROI^{ON}” regions detected over the course of an assay). As described herein, the inventors have demonstrated that the concentration of one or more analytes in a sample can be evaluated based on the number of recognition segments detected in a signal (see, e.g., Example 3 and FIGS. 10B-12D).

In some embodiments, the concentration of an analyte in a sample is determined based at least in part on a count of detected series of signal pulses in a single compartment of an array comprising a plurality of compartments. For example, in some embodiments, the method is performed in an arrayed format in which each compartment of an array is configured to contain therein an analyte as a single molecule. In some embodiments, the method comprises detecting at least one series of signal pulses, where each series of signal pulses is indicative of a series of binding events between one or more secondary reporters and an affinity reagent bound to a first analyte within a first compartment. In some embodiments, the concentration of the analyte in the sample is determined based at least in part on the count of the detected series of signal pulses of the first compartment. In some embodiments, an array comprises between about 10,000 and about 1,000,000 compartments. The volume of a compartment may be between about 10⁻²¹ liters and about 10⁻¹⁵ liters, in some implementations.

In some embodiments, the method comprises detecting at least one series of signal pulses in each of at least two compartments of the array. In some embodiments, the concentration of the analyte in the sample is determined based at least in part on the count of the detected series of signal pulses of the at least two compartments. As described herein, one or more characteristics of the detected series of signal pulses can be used to determine one or more chemical characteristics of an analyte, and thus, determine the identity of the analyte. Accordingly, in some embodiments, the detected series of signal pulses can be used to determine both the identity and concentration of an analyte in a sample.

In the context of an array, in some embodiments, the method comprises determining that two or more compartments of the array comprise an analyte of the same type. In some embodiments, the concentration of the analyte in the sample is determined based at least in part on the count of the detected series of signal pulses (ROIs) of the two or more compartments (e.g., an average of the detected series of signal pulses among the two or more compartments, a sum of the detected series of signal pulses (ROIs) among the two or more compartments). In some embodiments, two or more compartments of the array comprise analytes of a different type, and the method comprises determining a relative concentration of the analytes in the sample based at least in part on the detected series of signal pulses (ROIs) of the two or more compartments. In some embodiments, the relative concentration is determined based at least in part on a ratio of the count of detected series of signal pulses (ROIs) of one compartment to the count of detected series of signal pulses (ROIs) of another compartment. In other embodiments, the relative concentration is determined by the total ROIs across a plurality of compartments resulting from a first type of analyte relative to the total ROIs across a plurality of compartments resulting from a second type of analyte. In other embodiments, an absolute concentration is determined for each of the two types of analytes.

In some embodiments, the methods described herein comprise determining one or more chemical characteristics of an analyte (e.g., a polypeptide) based on one or more characteristics of at least one detected series of signal pulses.

In some embodiments, the one or more characteristics of the at least one detected series of signal pulses comprise a first recognition segment duration of a first series of signal pulses (e.g., a length of time during which the first series of signal pulses is detected). In some embodiments, the one or more characteristics of the at least one series of signal pulses comprise an average of two or more recognition segment durations. In some embodiments, a recognition segment duration is characteristic of a dissociation rate of affinity reagent binding. In some embodiments, recognition segment duration can be used to determine the identity of an affinity reagent, and thus, the analyte to which it is bound, based on known binding preferences among the one or more affinity reagents.

In some embodiments, the one or more characteristics of the at least one detected series of signal pulses comprise an intersegment duration between two recognition segment durations (e.g., a length of time between two successively detected series of signal pulses). In some embodiments, the one or more characteristics of the at least one series of signal pulses comprise an average of two or more intersegment durations. In some embodiments, an intersegment duration is characteristic of an association rate of affinity reagent binding. In some embodiments, intersegment duration can be used to determine the identity of an affinity reagent, and thus, the analyte to which it is bound, based on known binding preferences among the one or more affinity reagents.

In some embodiments, the one or more characteristics of the at least one detected series of signal pulses comprise a first pulse duration of a first series of signal pulses. In some embodiments, the first pulse duration comprises an average duration of pulses of the first series of signal pulses. In some embodiments, the first pulse duration is characteristic of a dissociation rate of secondary reporter binding. In some embodiments, pulse duration can be used to determine the identity of a secondary reporter, and thus, the affinity reagent to which it is bound, based on known binding preferences among the one or more secondary reporters. In some embodiments, pulse duration can thus be further indicative of an analyte to which the affinity reagent is bound based on known binding preferences among the one or more affinity reagents.

In some embodiments, the one or more characteristics of the at least one detected series of signal pulses comprise a first interpulse duration of a first series of signal pulses (e.g., a duration between successively detected signal pulses of the first series). In some embodiments, the first interpulse duration comprises an average duration between pulses of the first series of signal pulses. In some embodiments, the first interpulse duration is characteristic of an association rate of secondary reporter binding. In some embodiments, interpulse duration can be used to determine the identity of a secondary reporter, and thus, the affinity reagent to which it is bound, based on known binding preferences among the one or more secondary reporters. In some embodiments, interpulse duration can thus be further indicative of an analyte to which the affinity reagent is bound based on known binding preferences among the one or more affinity reagents.

In some embodiments, the at least one series of signal pulses comprises: a first set of at least one series of signal pulses indicative of a first series of binding events between one or more secondary reporters and a first affinity reagent bound to an analyte, and a second set of at least one series of signal pulses indicative of a second series of binding events between one or more secondary reporters and a second affinity reagent bound to the analyte. In some embodiments, the first affinity reagent is different from the second affinity reagent. In some embodiments, the first affinity reagent binds to a first site on the analyte, and the second affinity reagent binds to a second site on the analyte. In some embodiments, the first site does not comprise a post-translational modification (PTM), and the second site comprises a PTM. In some embodiments, the first site comprises a first PTM, and the second site comprises a second PTM.

In some embodiments, the methods comprise detecting at least one series of signal pulses in each of at least two compartments of an array, where the at least two compartments comprise a different proteoform of a polypeptide. In some embodiments, a plurality (e.g., two or more, three or more, four or more, five or more, ten or more) of compartments of the array comprise single polypeptide molecules corresponding to different proteoforms of a polypeptide. In some embodiments, the methods comprise detecting, in each compartment of the plurality, at least one series of signal pulses. In some embodiments, one or more characteristics in the detected series of signal pulses can be indicative of the specific proteoform in the compartment. Thus, in some embodiments, different characteristics in the series of signal pulses detected in different compartments can be used to distinguish between the different proteoforms of the polypeptide.

In some embodiments, an affinity reagent comprises a tag peptide configured for binding with a secondary reporter. For example, in some embodiments, the secondary reporter comprises a terminal amino acid recognizer (e.g., N-terminal amino acid recognizer), and the tag peptide comprises a free terminal amino acid (e.g., N-terminal amino acid) to which the recognizer binds. Thus, in some embodiments, each series of signal pulses is indicative of a series of binding events between one or more secondary reporters and the tag peptide of an affinity reagent bound to an analyte. In some embodiments, a tag peptide can be of any length suitable to permit binding of a secondary reporter to the tag peptide. In some embodiments, a tag peptide is at least two amino acids in length. In some embodiments, a tag peptide is up to 200 amino acids in length (e.g., 2-200, 2-100, 4-80, 5-50, 5-30, 5-20, 10-100, 20-80, 30-70 amino acids in length). In some embodiments, each of the one or more secondary reporters comprises a detectable label, such as a luminescent label (e.g., a fluorophore dye molecule).

As generally depicted in the schematic of FIG. 8B, in some embodiments, an analyte (e.g., a polypeptide) is attached to a surface through a capture reagent that binds the analyte. As shown, in some embodiments, the capture reagent binds to a site on the analyte that is different from a site to which an affinity reagent binds. In some embodiments, the capture reagent comprises an antibody, an antigen-binding portion of an antibody (e.g., a single-chain antibody variable fragment (scFv) or V_{H}H fragment), or an aptamer. In some embodiments, the affinity reagent comprises an antibody, an antigen-binding portion of an antibody (e.g., a single-chain antibody variable fragment (scFv) or V_{H}H fragment), or an aptamer.

In some aspects, methods described herein involving the use of affinity reagents and capture reagents can be performed without the use of secondary reporters. For example, in some aspects, the disclosure provides methods of sample analysis comprising: contacting a capture reagent with a sample comprising one or more analytes, where the capture reagent binds an analyte of the sample to form a first complex; contacting the first complex with one or more affinity reagents; detecting at least one series of signal pulses, where each series of signal pulses is indicative of a series of binding events between the one or more affinity reagents and the analyte; and determining a concentration of the analyte in the sample based at least in part on a count of detected series of signal pulses. Thus, in some embodiments, the methods permit direct detection of affinity reagent binding analyte, without the use of secondary reporters, to determine one or more recognition segment durations. In some embodiments, each of the one or more affinity reagents comprises a detectable label, such as a luminescent label (e.g., a fluorophore dye molecule).

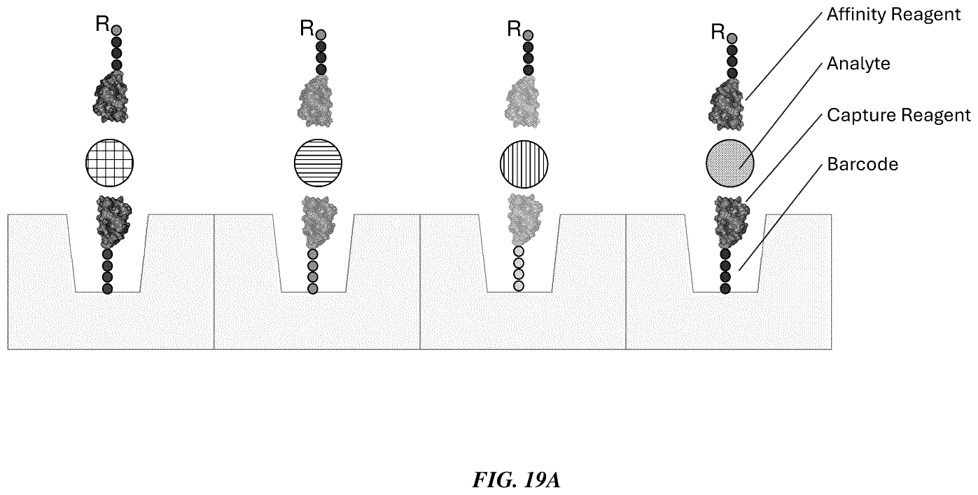

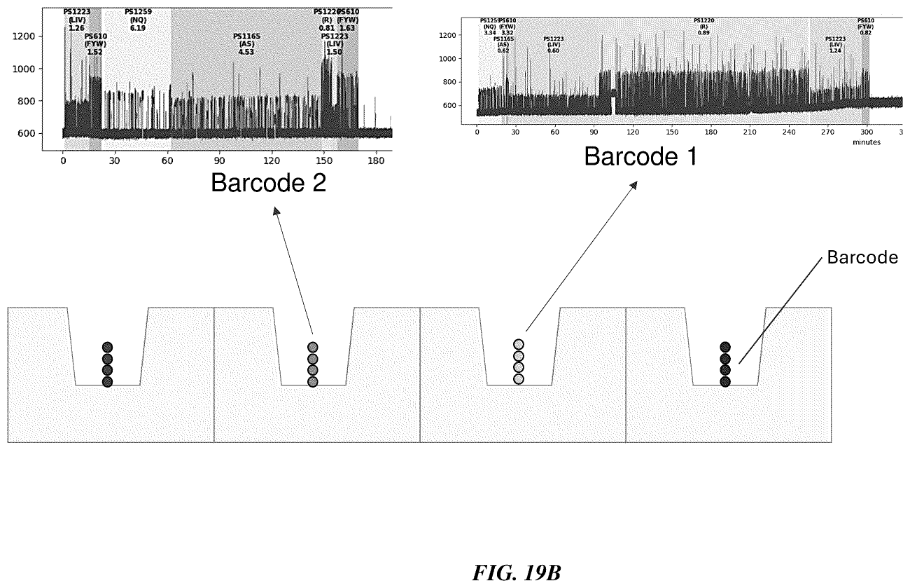

In some embodiments, the methods described herein can be performed in a multiplex format. In some embodiments, such methods can be multiplexed using barcodes (e.g., peptide barcodes), as illustrated by the example approach shown in FIGS. 19A-19B. As shown in FIG. 19A, different analytes are bound by different affinity reagents having tag peptides configured for recognition by the same secondary reporter (e.g., N-terminal arginine recognizer), and the secondary reporter signal can thus be used to identify a subset of analytes. Also as shown, the different analytes are further bound by different capture reagents having different barcodes, where the identity of each barcode can be used to identify the capture reagent to which it is conjugated (and thus, the specific analyte to which the capture reagent is bound). In some embodiments, as illustrated in FIG. 19B, the identity of the barcode can be determined by removing the capture reagent and sequencing the barcode. Thus, for example, in some embodiments, the barcode comprises a cleavage site between the barcode and the capture reagent to permit removal of the capture reagent prior to sequencing.

As described herein, in some embodiments, the disclosure provides methods that comprise contacting a capture reagent with a sample comprising one or more analytes, where the capture reagent binds an analyte of the sample to form a first complex, and contacting the first complex with one or more affinity reagents, where at least one affinity reagent binds the analyte of the first complex to form a second complex comprising the analyte, the capture reagent, and an affinity reagent. In some embodiments, the capture reagent is attached to a surface prior to forming the first complex with the analyte. In some embodiments, the methods comprise forming the first complex prior to attaching the first complex to a surface. In some embodiments, the methods further comprise forming the second complex prior to attaching the first complex to a surface.

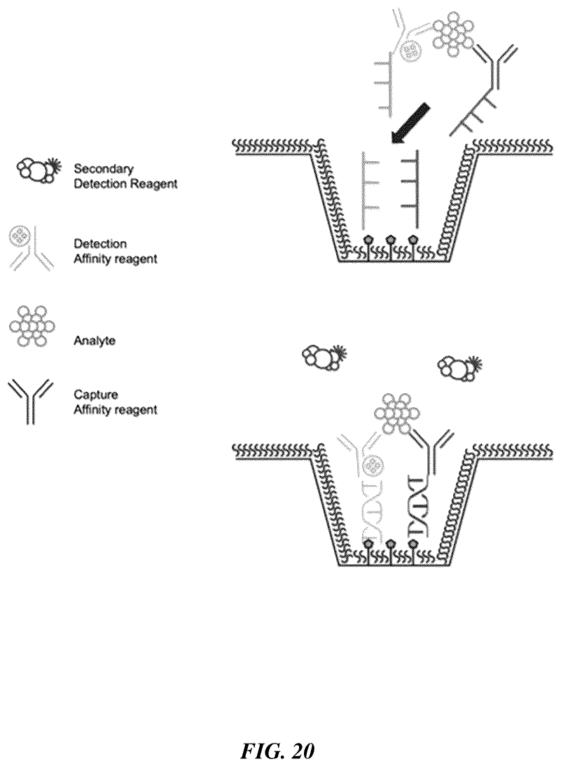

For example, FIG. 20 illustrates an example of conditional capture in which the second complex (e.g., comprising an analyte bound by each of a capture reagent and an affinity reagent) is attached to a surface only after the second complex is formed. As shown, in some embodiments, the capture reagent of the second complex is attached to the surface through a first linkage group, and the affinity reagent is attached to the surface through a second linkage group. In some embodiments, the capture reagent and the affinity reagent comprise a first and second oligonucleotide, respectively, and the surface comprises complementary oligonucleotides that hybridize to the first and second oligonucleotides to form the first and second linkage groups. In some embodiments, the capture reagent and the affinity reagent may bind weakly to the analyte individually but strongly together in the second complex, and the stronger interactions of the second complex would ensure both weak interactions between the reagents and the surface occur at the same time.

In some embodiments, the use of affinity reagents and capture reagents (e.g., as described herein and generally depicted in FIG. 8B) advantageously utilizes a sandwich-type configuration similar to well-known conventional methods of detection, such as an enzyme-linked immunosorbent assay (ELISA). This approach therefore advantageously permits the use of a vast library of binding reagents (e.g., antibodies) that are widely available and/or known in the art for targeting specific analytes. As would be appreciated by a person of skill in the art, such binding reagents can readily be implemented as affinity reagents and/or capture reagents in accordance with the methods described herein. Moreover, techniques for generating new binding reagents (e.g., antibodies) against an analyte of interest are similarly routine and well known to those skilled in the art.

Desirable characteristics of the reagents described herein would be apparent to those skilled in the art based on the present disclosure. For example, examples of suitable secondary reporters include amino acid recognizers, which have been described and characterized, for example, in PCT International Publication Nos. WO2020102741A1, WO2021236983A2, WO2023122769A2, WO2024031031A2, and WO2024086832A1, each of which is incorporated by reference in its entirety.

In some embodiments, desirable characteristics of an affinity reagent of the disclosure include, by way of example, an ability to bind an analyte with slower binding kinetics relative to the binding kinetics between a secondary reporter and the affinity reagent (e.g., such that the secondary reporter binds the affinity reagent at a faster rate than a time required for the affinity reagent to dissociate from the analyte). In some embodiments, desirable characteristics of an affinity reagent include, by further way of example, an ability to bind an analyte with a dissociation rate of binding (K_{D}) that is less than a dissociation rate of binding (K_{D}) between a secondary reporter and the affinity reagent.

In some embodiments, desirable characteristics of a capture reagent of the disclosure include, by way of example, an ability to bind an analyte with slower binding kinetics relative to the binding kinetics between an affinity reagent and the analyte (e.g., such that the affinity reagent binds the analyte at a faster rate than a time required for the capture reagent to dissociate from the analyte). In some embodiments, desirable characteristics of a capture reagent include, by further way of example, an ability to bind an analyte with a dissociation rate of binding (K_{D})) that is less than a dissociation rate of binding (K_{D}) between an affinity reagent and the analyte. In some embodiments, desirable characteristics of a capture reagent include, by further way of example, an ability to bind an analyte at a site on the analyte that is different from a site to which an affinity reagent binds.

In some embodiments, methods of dye cycling comprise the use of one or more post-translational modification-specific (PTM-specific) affinity reagents. For example, in some embodiments, the methods of dye cycling involve a single-molecule method comprising: (a) contacting a single polypeptide with an affinity reagent to produce a polypeptide-affinity reagent complex, optionally wherein the affinity reagent is an antibody that binds to the single polypeptide; (b) contacting the polypeptide-affinity reagent complex with one or more post-translational modification-specific (PTM-specific) affinity reagents; and (c) identifying whether the single polypeptide comprises a post-translational modification (PTM) by determining a luminescence signature representative of the binding interaction(s) between the polypeptide-affinity reagent complex and the one or more PTM-specific affinity reagents.

Such dye cycling methods can be useful in multiplexing affinity reagents. For example, in embodiments in which multiple (slow dissociation rate) affinity reagents are each tagged and recognized by a specific luminescent labeled secondary reporter (as shown in FIG. 7), there can be secondary reporters that have different dyes and different kinetics, leading to unique signal pulses. The slow kinetics of the affinity reagents can be observed as recognition segments which can be rather sparse with no detriment. In this configuration, the simultaneous binding of two affinity reagents will lead to pulsing by both of the corresponding secondary reporters. However, if the pulsing of each secondary reporter is reasonably sparse, pulsing events will be detected separately.

The use of secondary reporters can also help with the number of affinity reagents that can be used in a single experiment. In case of direct luminescent labeling, each affinity reagent must have a different dye (e.g., six different affinity reagents means six different dyes). However, with dye cycling methods, any given dye can be associated with multiple secondary reporters having different kinetics (thus, if N number of kinetic classes were possible, then 6×N number of different affinity reagents could be used).

A single molecule method could be particularly valuable when a collection of intact polypeptides/proteins are immobilized in different compartments on a chip and each of the proteins is individually characterized. This could be used, for example, to measure the representation of different proteoforms in a different sample. In some embodiments, the polypeptides can be immobilized covalently on a single chip. This can be achieved for example, using chemical modification of the polypeptides and subsequent immobilization on the chip. Each compartment would then correspond to a single protein, which would remain bound during the experiment. All of the affinity reagents and, possibly, secondary reporters would be in solution.

Another variant of a dye cycling method is one in which the intact polypeptides to be characterized are free in solution (i.e., not immobilized) and instead one of the affinity reagents is immobilized (e.g., on the surface of a chip). In such embodiments, the intact polypeptides would bind (reversibly) to each affinity reagent on the surface of the chip. While the intact polypeptide is bound to the chip via the affinity reagent, it can be characterized with further affinity reagents in solution. This might be advantageous in terms of throughput (e.g., the size of the population of polypeptides to be tested in a single experiment), sample prep, and/or sensitivity. Different affinity reagents bound to the chip may target different intact polypeptides in the sample and secondary reporters could identify which affinity reagents were immobilized in any given compartment of the chip.

### Post-Translational Modification-Specific (PTM-Specific) Affinity Reagents

A post-translational modification-specific (PTM-specific) affinity reagent is a molecule that binds to an amino acid comprising a post-translational modification (PTM). In some embodiments, the PTM-specific affinity reagent specifically binds to an amino acid comprising a PTM (e.g., binds to the amino acid having a PTM with a higher affinity than the same amino acid without the PTM).

PTM-specific affinity reagents include, for example, proteins and nucleic acids, which may be synthetic or recombinant. In some embodiments, a PTM-specific affinity reagent is an antibody (e.g., a single-chain antibody variable fragment (scFv) or VHH (Nanobody)). In some embodiments, a PTM-specific affinity reagent is an aptamer.

The PTM-specific affinity reagent can specifically bind to an amino acid comprising a phosphorylation (e.g., phospho-tyrosine, phospho-serine, or phospho-threonine), a glycosylation, acetylation (e.g., acetylated lysine), ADP-ribosylation, citrullination, formylation, (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), or ubiquitination (e.g., ubiquitinated lysine). In some embodiments, the PTM-specific affinity reagent can specifically bind to a phospho-tyrosine, phospho-serine, or phospho-threonine amino acid.

In some embodiments, a PTM-specific affinity reagent that specifically binds to phospho-tyrosine comprises an SH2 domain. In some embodiments, a PTM-specific affinity reagent that specifically binds to phospho-tyrosine is PS33.

In some embodiments, a PTM-specific affinity reagent specifically binds to a serine amino acid comprising a PTM. In some embodiments, a PTM-specific affinity reagent specifically binds to a threonine amino acid comprising a PTM. In some embodiments, a PTM-specific affinity reagent specifically binds to a tyrosine amino acid comprising a PTM. In some embodiments, a PTM-specific affinity reagent specifically binds to a lysine amino acid comprising a PTM. In some embodiments, a PTM-specific affinity reagent specifically binds to an asparagine amino acid comprising a PTM. In some embodiments, a PTM-specific affinity reagent specifically binds to a arginine amino acid comprising a PTM. In some embodiments, a PTM-specific affinity reagent specifically binds to a glycine amino acid comprising a PTM. In some embodiments, a PTM-specific affinity reagent specifically binds to a cysteine amino acid comprising a PTM. In some embodiments, a PTM-specific affinity reagent specifically binds to a methionine amino acid comprising a PTM.

### Amino Acid Recognition Molecules

An amino acid recognition molecule (e.g., terminal amino acid recognition molecule) is a molecule that specifically binds to a certain amino acid (e.g., binds to the certain amino acid with a higher affinity than any other amino acid). Amino acid recognition molecules include, for example, proteins and nucleic acids, which may be synthetic or recombinant. In some embodiments, an amino acid recognition molecule may be an antibody or an antigen-binding portion of an antibody, an SH2 domain-containing protein or fragment thereof, an FHA domain-containing protein or fragment thereof, or an enzymatic biomolecule, such as a peptidase, an aminotransferase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.” In some embodiments, an amino acid recognition molecule is an antibody (e.g., a single-chain antibody variable fragment (scFv) or VHH (Nanobody). In some embodiments, an amino acid recognition molecule is an aptamer.

In some embodiments, an amino acid recognition molecule is a degradation pathway protein. Examples of degradation pathway proteins suitable for use as recognition molecules include, without limitation, N-end rule pathway proteins, such as Arg/N-end rule pathway proteins, Ac/N-end rule pathway proteins, and Pro/N-end rule pathway proteins. In some embodiments, an amino acid recognition molecule is an N-end rule pathway protein selected from a Gid protein (e.g., Gid4 or Gid10 protein), a UBR box protein (e.g., UBR1, UBR2) or UBR box domain-containing protein fragment thereof, a p62 protein or ZZ domain-containing fragment thereof, a ClpS protein (e.g., ClpS1, ClpS2), Baculoviral inhibitor of apoptosis (IAP) repeat-containing (BIR) protein (e.g., BIR3), an Ntaq1 protein, and a Zer/Zyg protein.

Examples of amino acid recognition molecules and uses thereof, including methods of polypeptide sequencing and other sequencing reagents (e.g., cleaving reagents) suitable for use in accordance with the disclosure have been described more fully, for example, in PCT International Publication Nos. WO2020102741A1, WO2021236983A2, WO2023122769A2, WO2024031031A2, and WO2024086832A1, each of which is incorporated by reference in its entirety.

### Methods of Screening

The inventors have developed a novel methodology for screening of target protein modulators (e.g., protein inhibitors). In some embodiments, such methodologies are useful for identifying novel drug molecules (e.g., from a library of drug molecules) that modulate (e.g., inhibit) a target protein. These methods involve immobilization of either (a) compounds from a library of compounds, or (b) a target protein, to a chip array followed by observation of binding kinetics between the compounds of the library and the target protein in single-molecule experiments performed on the chip.

Some aspects of the disclosure provide a method of screening for modulators of (e.g., drugs that target) a target protein comprising: (a) contacting a chip array comprising a plurality of compartments with a library of different compounds; (b) immobilizing each of the different compounds of the library of different compounds to a surface of the chip array; (c) contacting the library of different compounds with a target protein; and (d) determining the luminescence signature representative of the binding interaction(s) between at least one different compound and the target protein, thereby identifying whether the at least one different compound is a modulator of the target protein. Further aspects provide a method comprising (a) contacting a chip array comprising a plurality of compartments with a target protein; (b) immobilizing the target protein to a surface of the chip array; (c) contacting the target protein with a plurality of different compounds; and (d) determining the luminescence signature representative of the binding interaction(s) between at least one different compound and the target protein, thereby identifying whether the at least one different compound is a modulator of the target protein.

The method may involve determination of a luminescence signature representative of binding interactions between each compound in a library and the target protein. These luminescence signatures may be representative of (and/or provide data relating to) the binding kinetics of each compound in a library and the target protein. For example, these luminescence signatures can enable determination of residence time of each compound for binding to the target protein, binding affinities between the compounds of the library and the target protein, and on-off kinetic rates of the compounds of the library with respect to the target protein. Determining these binding kinetic measurements that are representative of each compound in a library can allow for a rank-ordering of compounds within the library to identify the best (or most useful) compounds for targeting the desired target protein (e.g., for modulation, e.g., for inhibition).

Importantly, the methods of the present disclosure as described herein, enable the use of a single-molecule screening platform, as the experiment can be designed such that each compartment of a chip array contains only a single molecule (e.g., a compound of a library) immobilized to its surface. Thus, these methods allow for efficient screening of a large number of compounds from a library when using a single chip array (e.g., a chip array having 96, 384, or 1536 compartments) by providing single-molecule data for each compound within an experiment.

In embodiments in which compounds of a library are immobilized to the compartments of the chip array, the compound belonging to a certain compartment (e.g., a compartment in which the unknown compound demonstrated high affinity for a target protein) can be rapidly identified using a sequencing step following observation of binding kinetics between the compounds and the target protein. For example, a sequencing step may comprise contacting each of the different compounds with one or more terminal amino acid recognition molecules, wherein the terminal amino acid recognition molecule recognize either the compound itself (e.g., in embodiments in which the compound is a peptide) or a barcode linked to the compound (e.g., in embodiments in which the compound is a small molecule); and detecting a series of signal pulses indicative of association of the one or more terminal amino acid recognition molecules with successive amino acids exposed at a terminus of a peptide (e.g., the compound or a barcode linked to a small molecule compound) while each peptide is being degraded, thereby sequencing each peptide. This combination of the initial screening step (e.g., observing binding kinetics between the compounds of the library and the target protein) with a sequencing step can be done on the same chip array and within the same instrument, thus increasing the efficiency of the screening platform.

A library of different compounds (e.g., a library of peptides) may comprise at least two, at least five, at least 10, at least 20, at least 30, at least 50, at least 75, at least 100, at least 200, at least 250, at least 500, or at least 1000 different compounds. In some embodiments, a library of different compounds (e.g., a library of peptides) comprises 2-10, 5-50, 20-50, 25-75, 50-100, 50-250, 100-500, 200-750, 300-800, 750-1000, or 750-2000 different compounds.

The luminescence signature observed regarding interactions between each of the different compounds in a library and the target protein can result from a luminescent label or conductivity label that is present on a molecule within the binding interaction. In some embodiments, the luminescent label or conductivity label is linked to the target protein. In other embodiments, the luminescent label or conductivity label is linked to each of the different compounds in a library.

### EXAMPLES

### Example 1

This Example describes an exemplary method of the disclosure for use in identifying the presence of a phosphorylated tyrosine on a model polypeptide.

A control polypeptide (LAQYLAYPDDDK) and a model polypeptide comprising a phospho-tyrosine (LAQ-pY-LAYPDDDK) were tested in this Example. Each polypeptide was first immobilized onto independent surfaces of a chip. A post-translational modification-specific (PTM-specific) affinity reagent that binds to phospho-tyrosine (PS33) and comprised a fluorescent label was then added to the chip and allowed to contact the polypeptides. A fluorescence signature representative of the binding interaction(s) between each polypeptide and the PTM-specific affinity reagent was collected using a detector for 30 minutes. The chip was then washed to remove the PTM-specific affinity reagent. A protein sequencing analysis of the polypeptides was then performed. A mixture of labeled terminal amino acid recognition molecules was added to the chip and allowed to incubate for 15 minutes. After 15 minutes, a mixture of cleaving reagents (a mixture of aminopeptidases) was added to the chip. During these steps, a series of signal pulses indicative of association of the one or more terminal amino acid recognition molecules with successive amino acids exposed at a terminus of each polypeptide were collected while each polypeptide was being degraded.

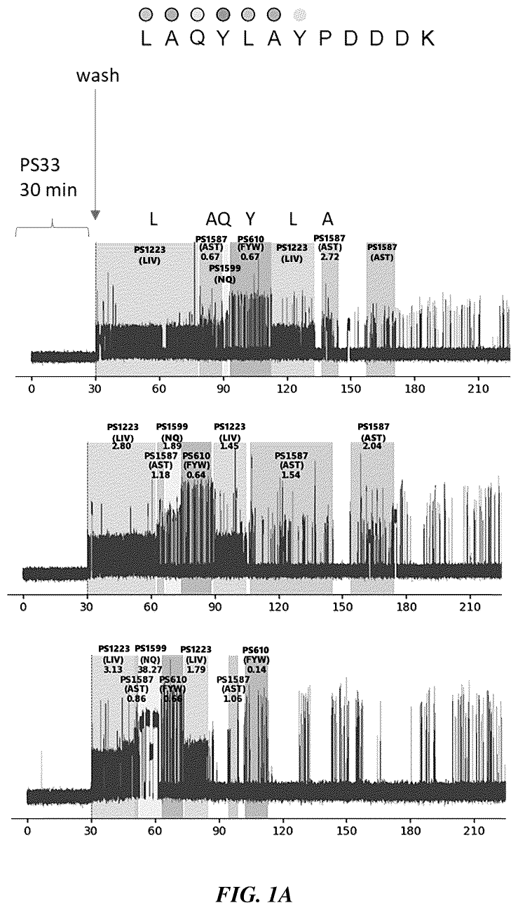

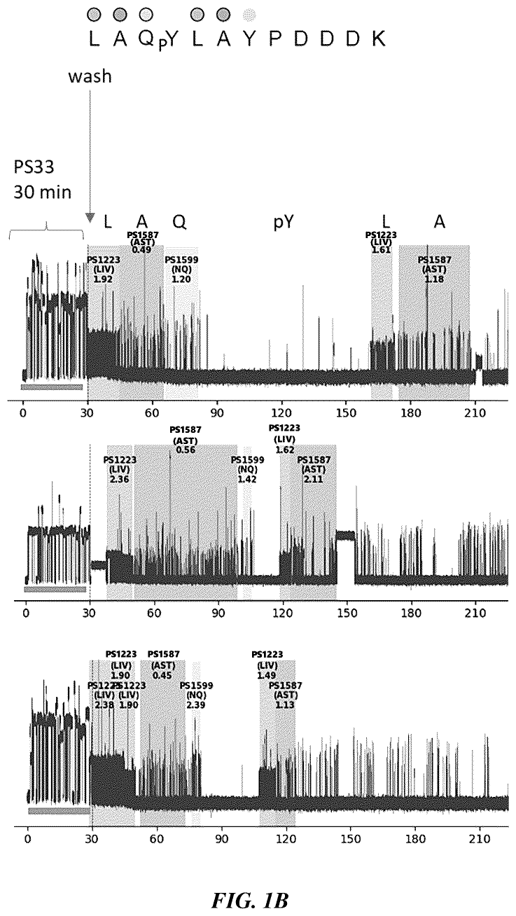

As shown in FIG. 1A, there was no fluorescence signature representative of binding interaction(s) between the polypeptide that did not include a PTM (LAQYLAYPDDDK) and the PTM-specific affinity reagent. On the contrary, there was a strong fluorescence signature representative of binding interaction(s) between the polypeptide that did include a PTM (LAQ-pY-LAYPDDDK) and the PTM-specific affinity reagent (FIG. 1B). In addition to identifying that the model included a phospho-tyrosine, the inventors were further able to determine that it was the more N-terminal tyrosine (i.e., the tyrosine at position 4 of the polypeptide) that was phosphorylated and not the second tyrosine (i.e., the tyrosine at position 7) based on the sequencing analysis. Note that, in this context, the sequencing analysis alone would not have identified that the amino acid at position 4 was a phospho-tyrosine. The terminal amino acid recognition molecule for tyrosine did not identify the amino acid at position 4 as being a tyrosine.

### Example 2

This Example demonstrates that methods of the disclosure can be used to identify a single post-translational modification within the context of a full-length natively folded protein. Such methods can be useful for determination of proteoforms in a sample (e.g., determination of proteoforms at a single-molecule level).

A full-length polypeptide (green fluorescent protein (GFP)) was immobilized on a surface of a chip. A PTM-specific affinity reagent (a fluorescently labeled VHH nanobody that binds to a PTM) was added to the chip and allowed to interact with the full-length polypeptide under native conditions. The PTM-specific affinity reagent was added to a chip containing no immobilized polypeptide as a control experiment. A fluorescence signature representative of the binding interaction(s) between the polypeptide and the PTM-specific affinity reagent was collected using a detector for 30 minutes.

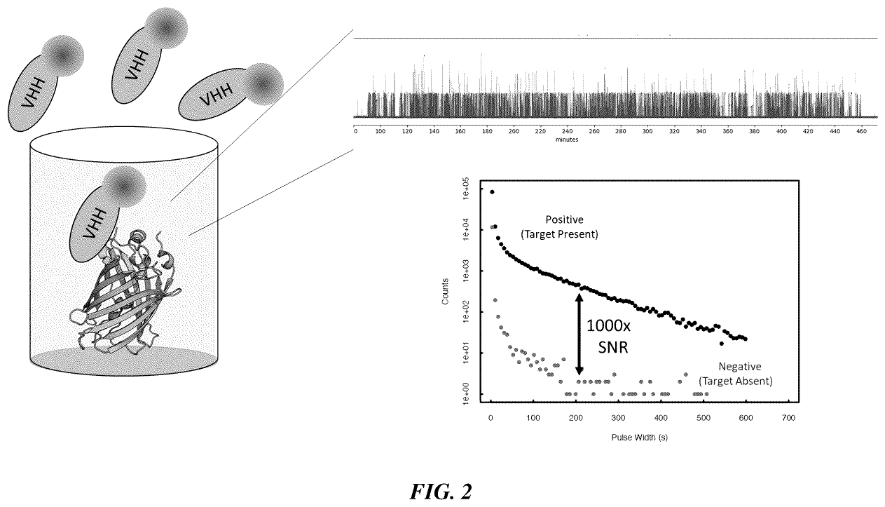

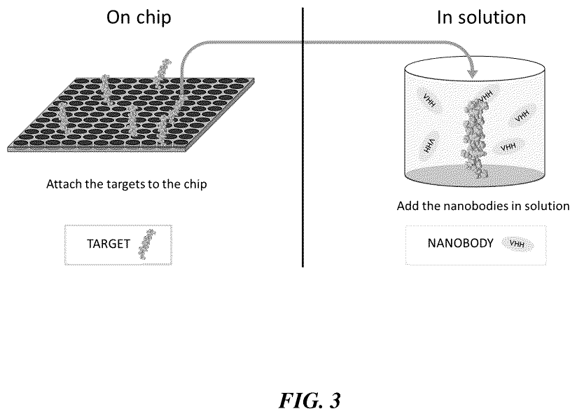

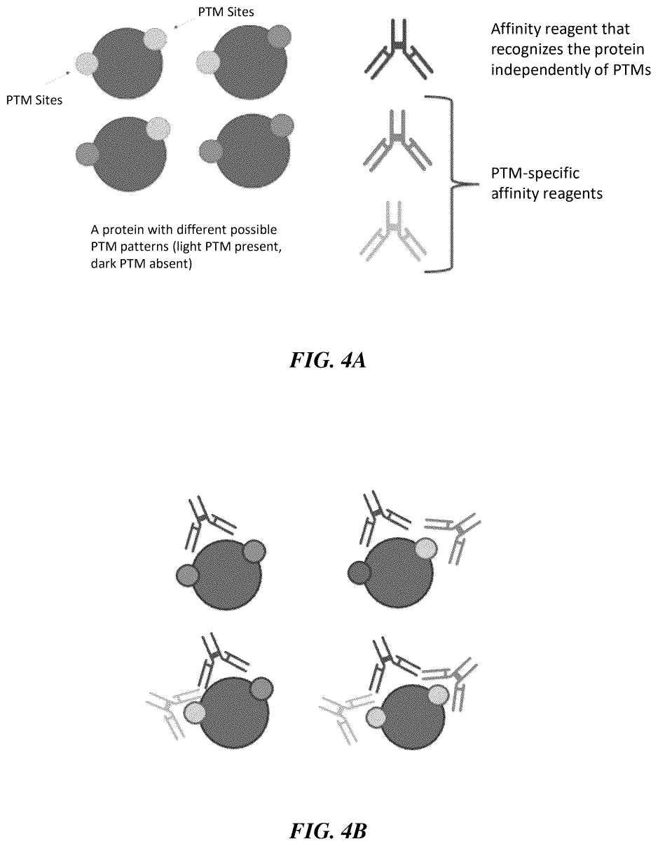

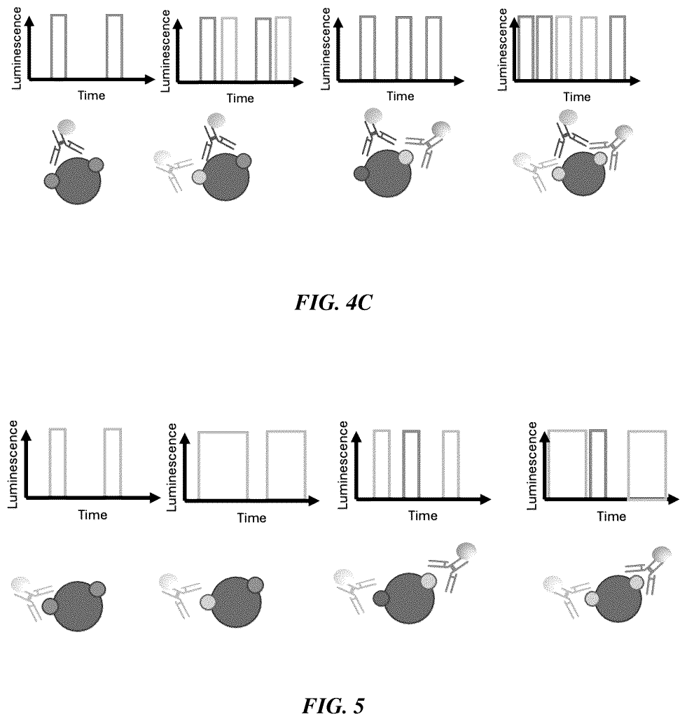

As shown in FIG. 2, there was a strong fluorescence signal detected in the experimental condition including the full-length polypeptide and the labeled PTM-specific affinity reagent. Conversely, there was minimal signal detected in the control containing only the labeled PTM-specific affinity reagent. The difference in signal detected provided a signal-to-noise ratio of approximately 1000, indicating that the method was very robust in its ability to determine that the full-length polypeptide comprised a PTM.

### Example 3. Dye Cycling for Highly Sensitive Protein Detection

### BACKGROUND

Genomics and proteomics are complementary descriptions of the subcellular level of organisms. While the genome codes for what governs the structure, function, and behavior of cells, the proteome captures what is really happening rather than just relying on gene expression levels. Proteins dictate cellular structure and activity, provide the mechanisms for signaling between cells and tissues, and catalyze chemical reactions that support metabolism. Their structure implies the function or the dysfunction. Thus, proteins can be the root cause of diseases (such as Alzheimer's or Huntington's disease), but they can also be used to cure it (for instance, antibodies are proteins).

Humans have around 20,000 protein-producing genes. The analysis of protein mixtures is particularly challenging due to the translation of these genes into a diversity of “proteoforms”, by which we refer to all of the different molecular forms in which the protein product of a single gene can be found. This variability can come from post-translational modifications occurring after the synthesis of the protein, but it can also come directly from genetic variants (germline) or transcription through alternative splicing (isoforms). For instance, human hemoglobin is present in red blood cells and one of its proteoform is the haemoglobin A1c that can get glycosylated after exposure to glucose in the blood. This glycosylated form of the protein has been used as a biomarker to detect multiple diabetes. Proteomics methods are grouped in two categories; the bottom-up (BU) approach, used in mass spectrometry (MS), which is based on protein fragmentation, and the top-down (TD) approaches that analyze intact proteins, which is only possible for smaller proteins (<70 kDa) with MS or other approaches from the classic ELISA to more recent next-generation assays.

In preliminary experimental work, recombinantly expressed proteins were immobilized on the surface of a chip array, and the ability to reliably detect antibody/protein interactions on the chip environment was demonstrated using three different model systems: GFP protein with an anti-GFP nanobody, Lysozyme protein with an anti-Lysozyme immunoglobulin, and Lysozyme protein with two anti-Lysozyme nanobodies. Despite showing the ability to recognize full proteins at the single molecule level by fluorescent imaging, the fluorescent signal obtained was insufficient to characterize the protein-protein interactions. Without wishing to be bound by any particular theory, these observations were attributed to photobleaching effects related to the short lifetime of the dyes compared to the long lifetime of antibody-target interactions. Indeed, the fluorescent signal reporting the IgG-protein interaction was not reporting the entire duration of the binding, causing a loss of essential information about the recognition features resulting in misinterpretation of its kinetic properties. Moreover, in this configuration, a binding event would be detected as a single fluorescent pulse, which may be difficult to distinguish from the background.

### Dye Cycling

To overcome the limitations attributed to photobleaching, a new approach referred to as “dye cycling” was developed to detect long-lived interactions at the single-molecule level. In this approach, as generally depicted in FIGS. 8A-8B, an N-terminal reporter peptide is added to the affinity reagent (e.g., antibody) by chemical modification, genetically, or other suitable means. This peptide is designed to be recognized by an N-terminal amino acid (NAA) recognizer with favorable fast kinetics, many examples of which have been described. The peptide-labeled affinity reagents are then used to detect surface-immobilized analytes (e.g., proteins), which can be immobilized directly to a surface or through a surface-immobilized capture reagent (e.g., antibody) that binds the analyte. In these conditions, the formation of the complex between the peptide-labeled affinity reagent and analyte will entail the transient immobilization of the peptide label. In the presence of NAA recognizer(s), the peptide (and thus, the analyte-bound affinity reagent) is detected via a series of pulses with specific kinetics. The pulsing will then stop when the analyte/affinity reagent interaction is disrupted. The continuous fluorescent signal reports the duration of the affinity reagent bound to the analyte and is referred to as ROI^{ON}, the duration of which is inversely proportional to the k_{off} of the affinity reagent. The duration of time between two analyte/affinity reagent binding events is referred to as ROI^{OFF} and is inversely proportional to the kₒₙ of the affinity reagent.

In contrast to direct labelling of the affinity reagent, the dye cycling methodology creates a very specific recognition pattern, specificity being conferred by tailoring the interaction between the reporter peptide and the NAA recognizer. Moreover, the fluorophore used to detect the interaction is continuously replaced, avoiding photobleaching and greatly increasing the dynamic range over which kinetic measurements are possible.

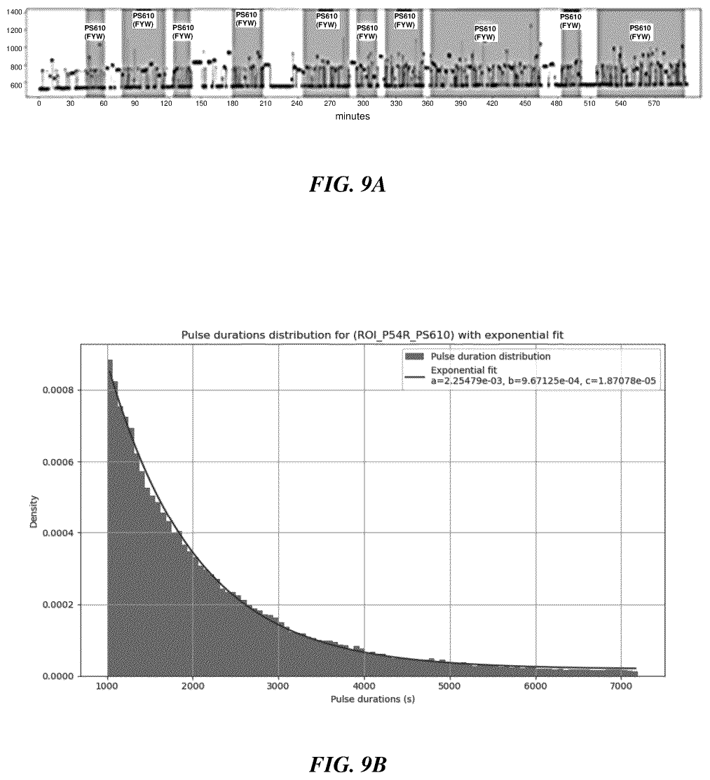

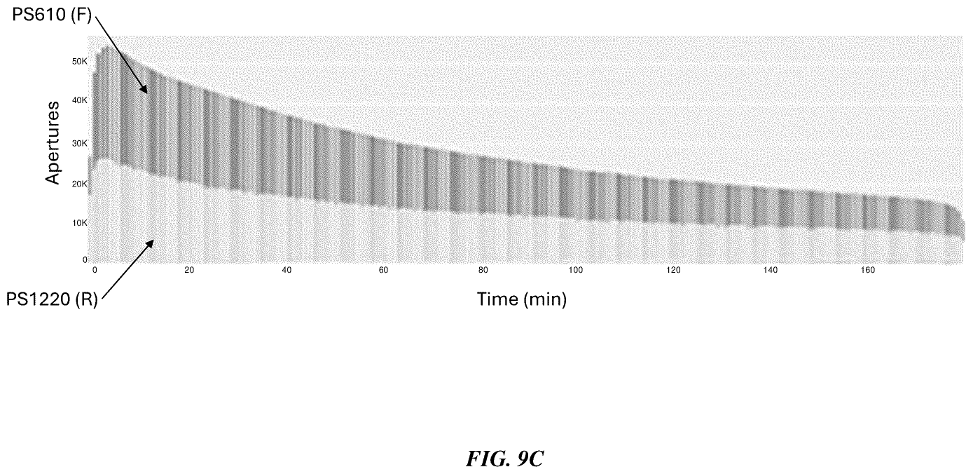

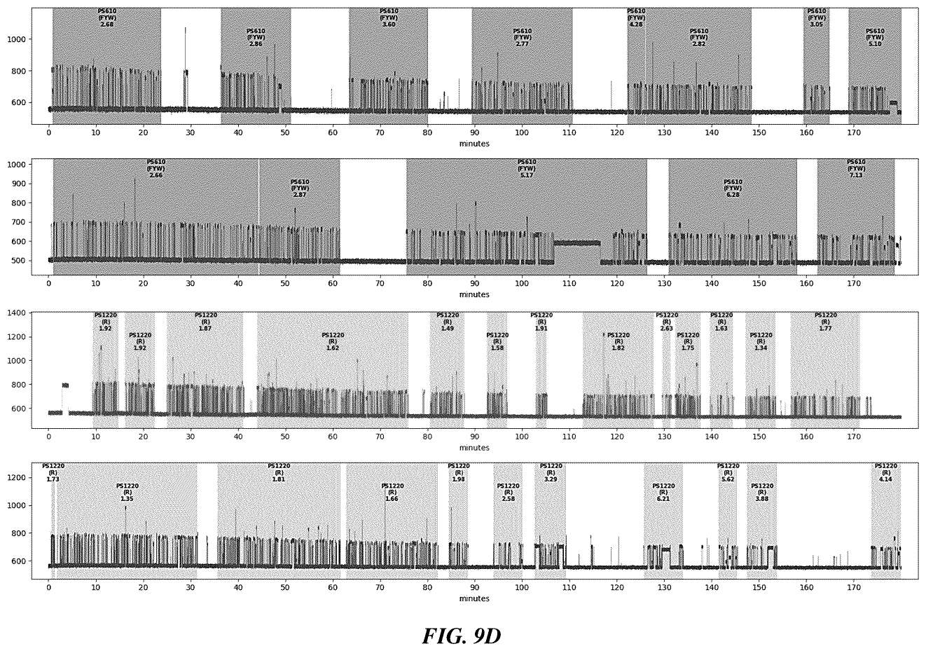

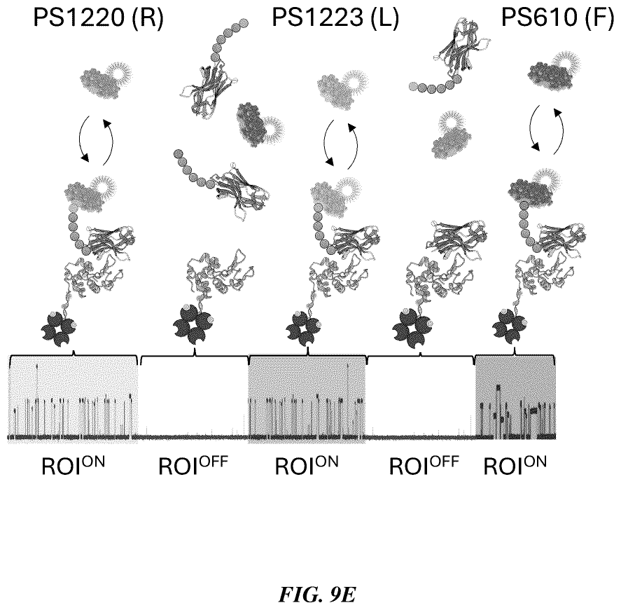

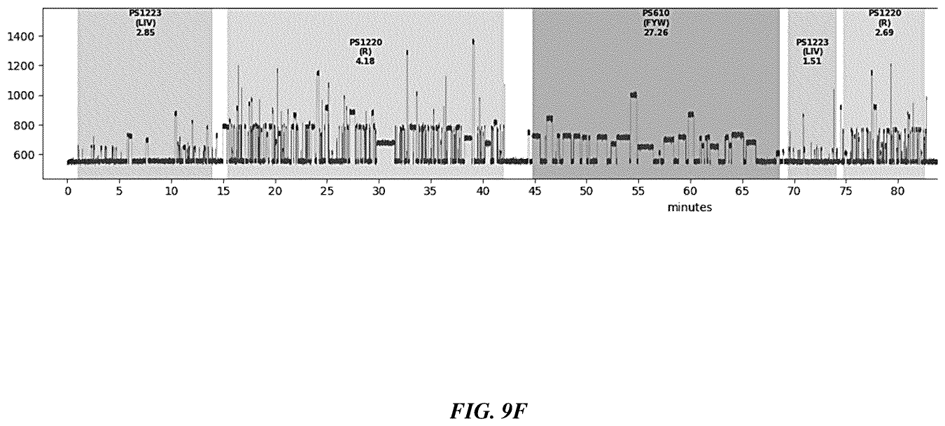

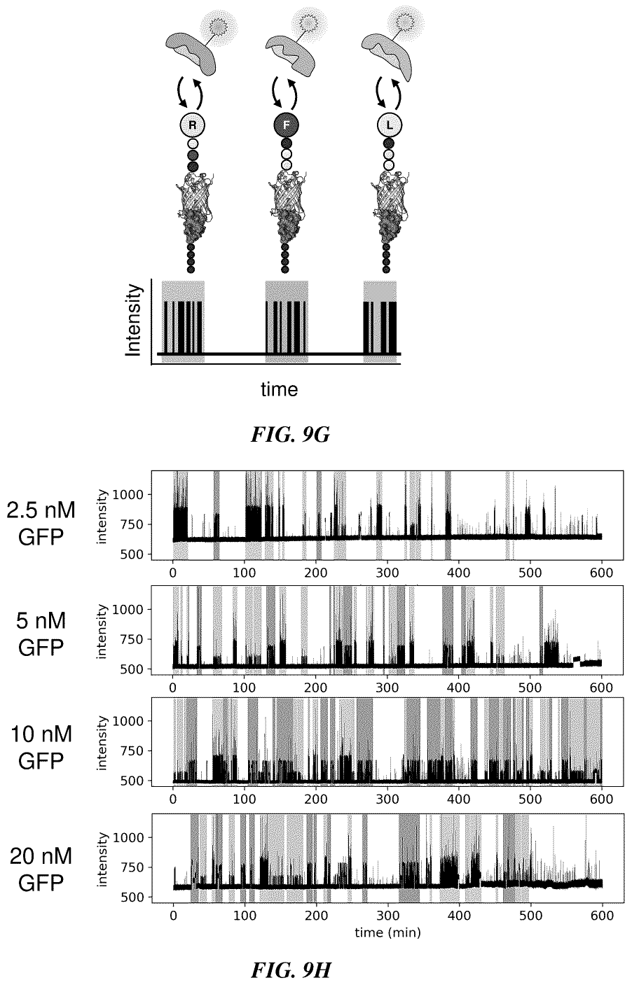

The dye cycling approach was evaluated using GFP and lysozyme analytes with anti-GFP VHH and anti-lysozyme VHH affinity reagents. Specific recognition of GFP and lysozyme was evaluated separately and in a mixture of GFP: Lysozyme with a 1:1 ratio, demonstrating the ability of distinguish multiple proteins in a single recognition experiment. The results further demonstrated accurate measurement of kinetic rates up to 10⁻⁴ s⁻¹. Representative experimental results are shown in FIGS. 9A-9D and described below.

FIG. 9A shows the kinetic signature of the anti-Lysozyme VHH F-1ZV5, labeled with N-terminal phenylalanine peptide, to the Lysozyme target protein in a single aperture as reported by the recognition of the N-terminal phenylalanine by PS610. As shown by the shaded regions, interaction between the anti-lysozyme and lysozyme target protein creates a distinct ROI^{ON}. The fluorescent signal intensity of the recognizer is represented in the y axis. The duration of the recognition run in minutes is represented in the x axis. FIG. 9B is a plot showing exponential decay of the distribution of the specific ROI^{ON} durations with a simple exponential fit (a*exp(−b/t)+c), which allows K_{off} determination (with b=k_{off}=9.5 10⁻⁴ s⁻¹). This value is in accordance with the scientific literature for this VHH (Genst et al. 2006).

FIG. 9C shows results demonstrating recognition of GFP and Lysozyme in a single run with GFP: Lysozyme in a 1:1 ratio in mixture with peptide-labeled affinity reagents (anti-GFP R-Lag16 VHH, anti-Lysozyme VHH F-1ZV5) and recognizers (PS1220 (R), PS610 (F)). Multiple ROI^{ON} were observed for R-Lag16 VHH and F-1ZV5 VHH. FIG. 9D shows representative kinetic signatures of R-Lag16 (bottom two panels) and F-1ZV5 (top two panels) in independent apertures containing cither GFP or lysozyme immobilized.

In another set of experiments, as generally illustrated in FIG. 9E, the anti-Lysozyme VHH 1ZV5 was labeled with peptide having either N-terminal F, L, or R amino acids. The differently labeled affinity reagents were combined and evaluated in a mixture with lysozyme and recognizers. FIG. 9F shows a representative kinetic signature of the differently labeled anti-Lysozyme VHHs binding the Lysozyme target protein in a single aperture. In the context of a mixture of affinity reagents, the binding events between single VHH molecules to the Lysozyme were observed independently, as reported by different recognizers (PS1223 for VHH having N-terminal L; PS610 for VHH having N-terminal F; and PS1220 for VHH having N-terminal R). Similar work was conducted using an immobilized anti-GFP in a mixture with recognizers and GFP labeled with peptide having either N-terminal F, L, or R amino acids, as generally illustrated in FIG. 9G. FIG. 9H shows representative kinetic signatures of anti-GFP binding the differently labeled GFP molecules in mixtures with 2.5, 5, 10, or 20 nM GFP.

### Dye Cycling Sandwich Assay

As a proof-of-concept of an immuno-sandwich assay (FIG. 8B), two anti-lysozyme nanobodies were utilized: D2-L29 (1ZV5) and D2-L24 (1ZVH), which are reported to have nanomolar affinity for two different epitopes on chicken egg white lysozyme and to bind simultaneously to lysozyme (Genst et al. 2006). The higher affinity nanobody, D2-L29, was chosen to be the capture reagent and was immobilized to the chip surface. The lower affinity nanobody, D2-L24, was produced with a peptide label carrying an N-terminal R to permit recognition by PS1220. FIG. 10A shows a scheme representing the dynamic sandwich assay for lysozyme quantification with a capture reagent (1ZV5 VHH), an affinity reagent labeled with N-terminal R for detection (R-1ZVH VHH), and a secondary affinity reagent reporting the sandwich (PS1220 recognizer). Representative experimental results are shown in FIGS. 10B-10D and described below.

FIG. 10B shows representative kinetic signatures obtained in mixtures having 1 ng/mL lysozyme (top panel) or 1 pg/mL (bottom panel) lysozyme. At high concentrations, traces show multiple binding event per aperture. At low concentrations at most one binding event is seen per aperture and, to assess analyte concentration, it is necessary to collect binding events from multiple apertures. FIG. 10C shows 2-dimensional histograms of measured average Pulse Width (PW) and average Inter Pulse Duration (IPD) for ROIs in mixtures having 10 ng/mL lysozyme (top panel), 100 pg/mL lysozyme (middle panel), or no lysozyme as control (bottom panel). At high concentration, the vast majority of the observed kinetic signatures represent binding events. These are characterized by a mean PW and mean IPD falling within a narrow range (red box). At lower concentrations we observe the same population, again falling into the range denoted by the red box, plus a second population corresponding to background noise. The knowledge of the expected PW and IPD allows filtering of the background, by only counting the ROIs inside the red box. Other ROI features could also possibly also help in the filtering. ROIs falling outside the red box are measured also in the absence of the analyte confirming their identification as noise. FIG. 10D is a titration curve for lysozyme showing response in number of ROI per concentration of analyte and concentration of Lysozyme in pg/mL. The results collectively demonstrated that the number of detected ROIs can be used to determine the concentration of analyte in a sample. This includes, for example, use of the number of detected ROIs in a single aperture (e.g., for higher analyte concentrations) and/or use of the number of detected ROIs across multiple apertures of an array of apertures (e.g., for lower analyte concentrations). Moreover, the detection methodology proved to be highly sensitive, allowing for analyte detection at very low concentrations which are physiologically relevant.

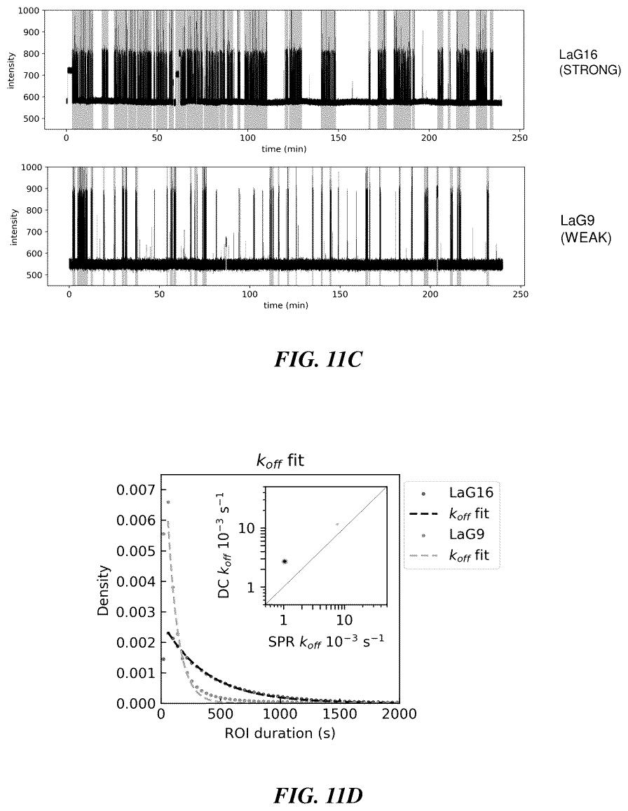

In another set of experiments, GFP labeled with a N-terminal arginine peptide (R-GFP) as analyte was evaluated with Lag16 as capture antibody and PS1220. FIG. 11A depicts a general scheme of this approach and illustrates the relation between number of detection events (ROI^{ON}; shaded regions) and analyte binding events to the capture antibody. When the R-GFP is captured into the aperture by Lag16, PS1220 reports its presence with rapid pulsing, creating an ROI^{ON}; when the GFP is not bound, no PS1220 is observed, creating ROI^{OFF} regions in the pulsing profile. FIG. 11B is a titration curve for R-GFP as analyte showing response in number of ROI per concentration of analyte and concentration of GFP in pg/mL. FIGS. 11C-11D show comparative results from these studies using either Lag16 or Lag9 as capture reagent. As shown, slower dissociation kinetics as measured for Lag16 give rise to longer ROI durations as compared to Lag9.

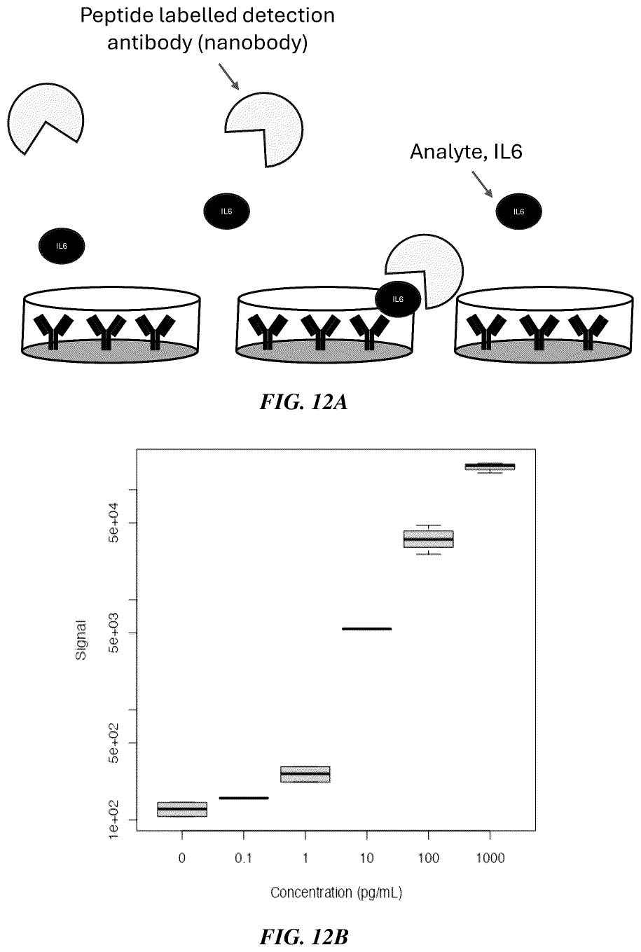

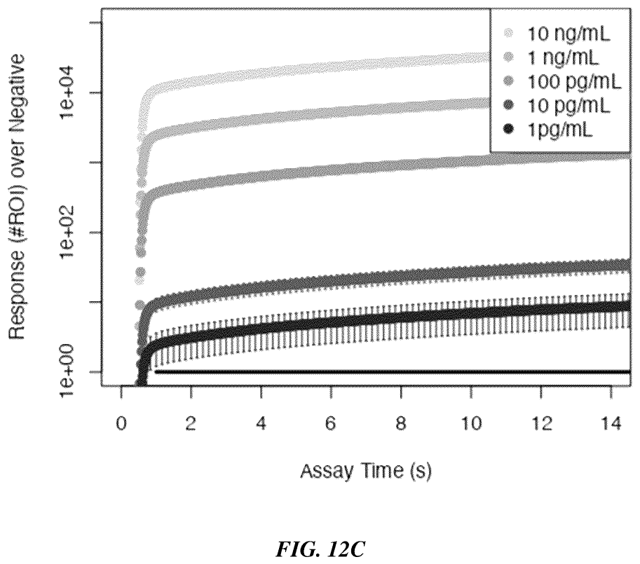

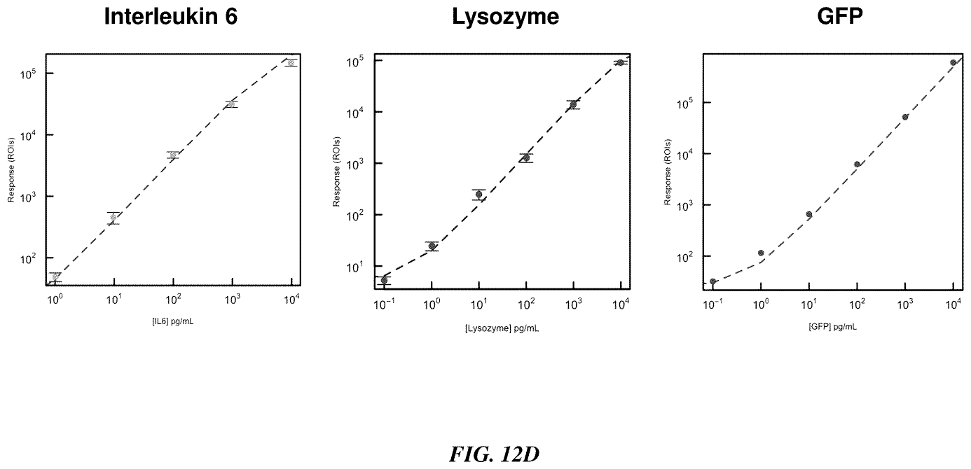

The use of antibody capture reagents was evaluated using IL-6 as analyte, as generally illustrated in FIG. 12A. These experiments were carried out in a mixture with IL-6 analyte, a biotinylated IgG as the capture reagent, a peptide-labeled nanobody (VHH) as the detection reagent, and a recognizer that binds the peptide label. The biotinylated antibody capture reagent is immobilized onto the chip surface via the biotin-streptavidin interaction. FIGS. 12B-12C show example results from runs carried out at different IL-6 concentrations.

IL-6 analyte was further evaluated, alongside lysozyme and GFP analytes, in spike-in titration experiments in serum. The results from these experiments (titration curves shown in FIG. 12D) demonstrated detection in serum at concentrations of about 0.1-1 pg/mL.

### Recognition and Isoforms Discrimination

Assays that characterize the alternative proteoforms of a protein present in a sample are gaining prominence in diagnostics. However, it is difficult to obtain accurate and reliable results using standard proteomic approaches based on fragmentation because association between multiple, distal modifications can be lost in the fragmentation process, which erases important biological information. This is the case for the microtubule associated protein Tau, involved in Alzheimer's, where association between splicing variants and phosphorylation is of critical importance.

Tau is a microtubule-associated protein encoded by the MAPT gene, located on the short arm of chromosome 17. In the human central nervous system, alternative splicing of the MAPT gene produces six tau isoforms, which vary in the number of N-terminal inserts (0N, 1N, or 2N) and C-terminal repeat domains (3R or 4R). These isoforms are differentially expressed depending on brain region and developmental stage. Additionally, an alternative isoform, known as Big Tau, results from exon 4a inclusion and is primarily expressed in the peripheral nervous system, increasing tau's molecular weight from approximately 45-65 kDa to 110 kDa.

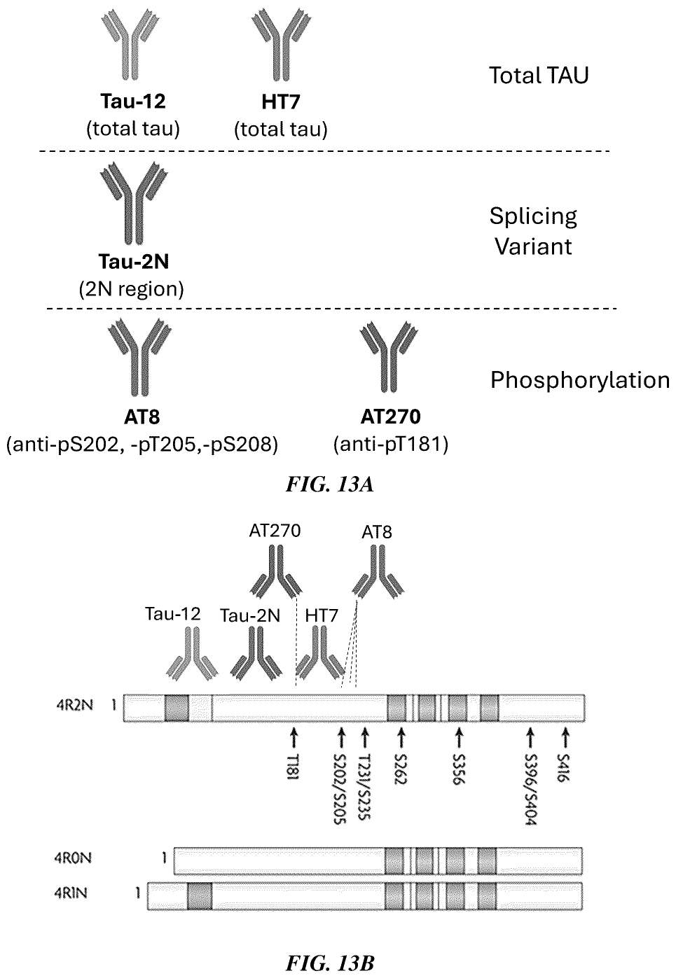

The dye cycling sandwich approach was further evaluated in the context of isoform discrimination among the Tau isoforms 0N and 2N or 3R and 4R. FIGS. 13A-13B show anti-Tau IgGs that were utilized as affinity and/or capture reagents in these studies: Tau-12 and HT7 for binding multiple Tau isoforms independent of modification, Tau-2N for specific binding to 2N isoform, and AT8 and AT270 for binding specific post-translational modifications (phospho-PTMs).

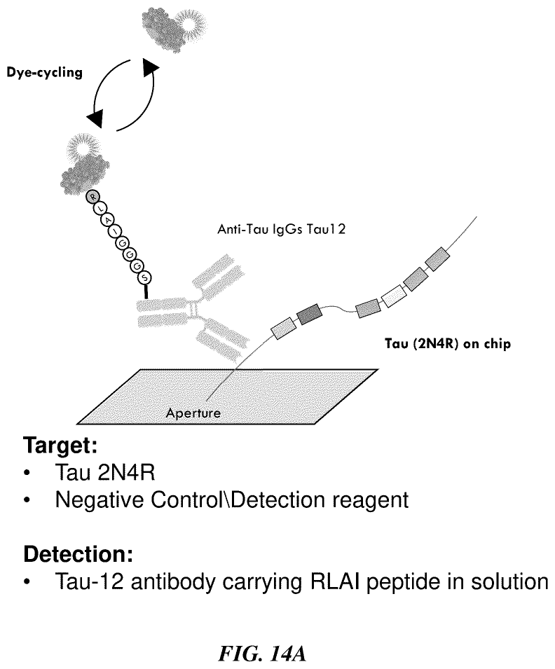

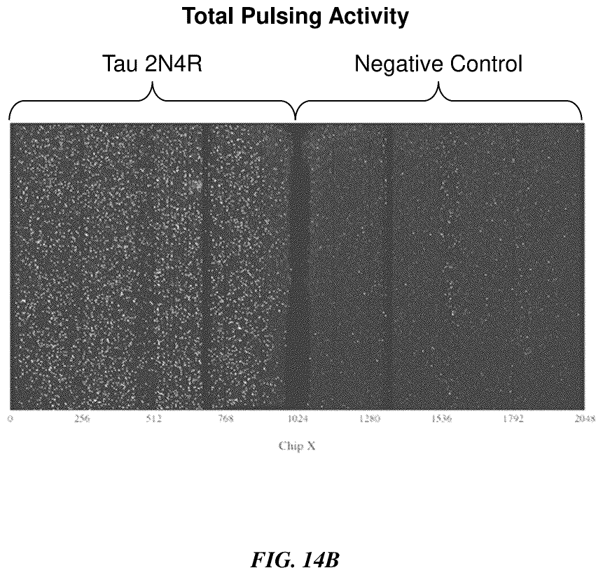

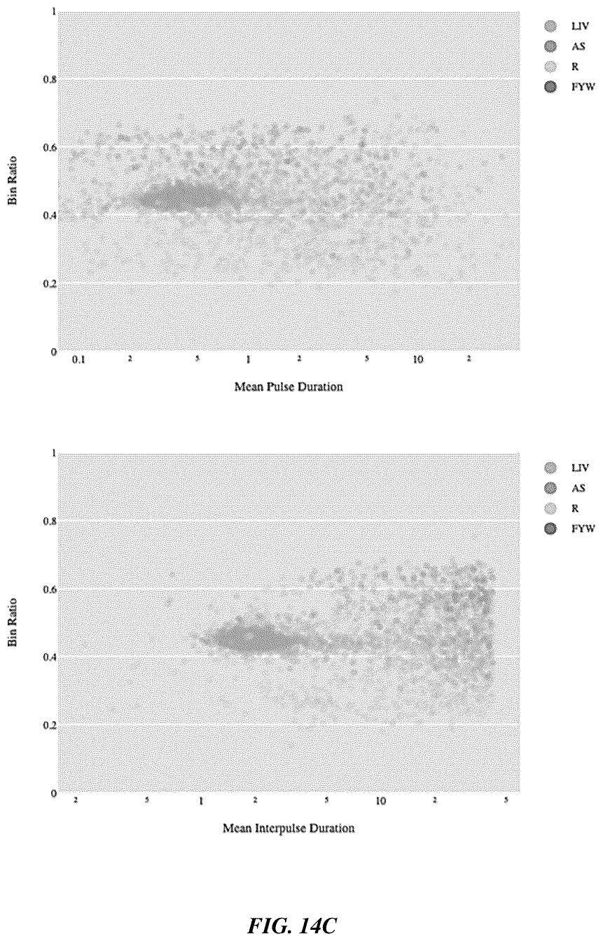

In one set of experiments, the Tau isoform 2N4R (0.2 nM) was immobilized directly on chip in mixture with Tau-12 antibody (25 nM), which carried a peptide label having N-terminal arginine, and the arginine recognizer PS1220 (FIG. 14A). As shown in FIG. 14B, the chip showed pulsing activity indicative of 2N4R recognition (negative control: GFP (0.2 nM) immobilized on chip in place of Tau). To enable discrimination of fluorescent dye labels by fluorescence lifetime and intensity, the chip rapidly alternates between early and late signal collection windows associated with each laser pulse, thereby collecting different portions of the exponential fluorescence lifetime decay curve. The relative signal in these collection windows (termed “bin ratio”) provides a reliable indication of fluorescence lifetime. FIG. 14C shows representative plots of bin ratio as a function of mean pulse duration (top plot) or mean interpulse duration (bottom plot) based on kinetic data obtained in these runs. This data shows how recognition of Tau isoform 2N4R produces ROIs with well-defined properties.

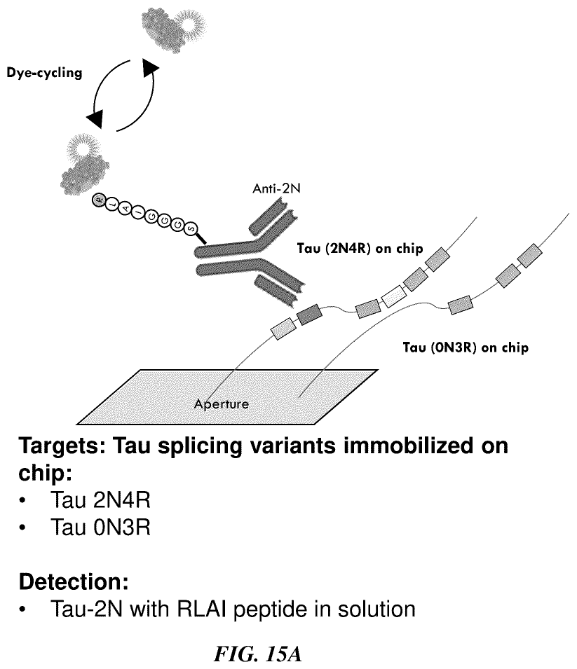

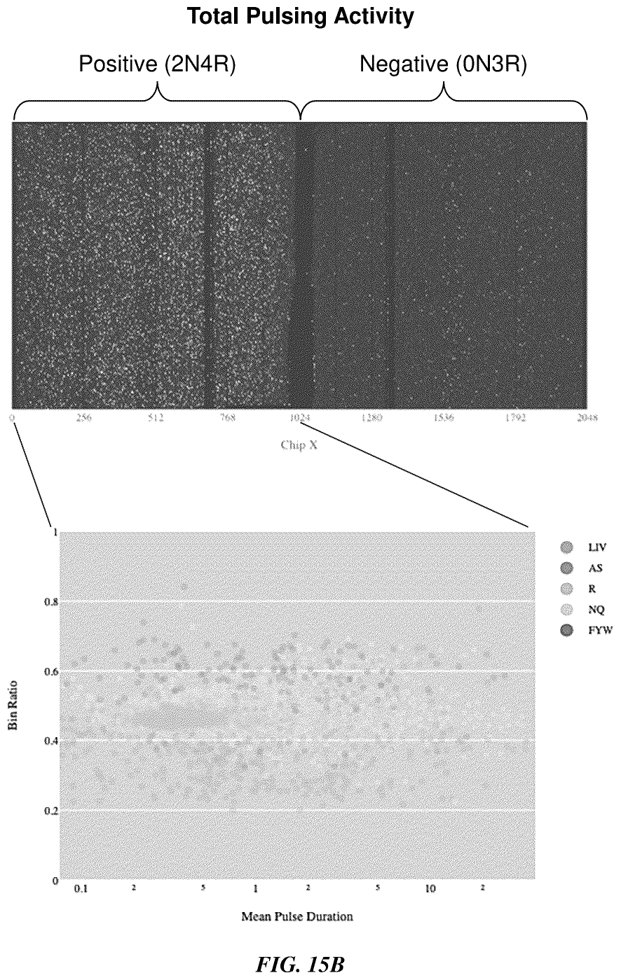

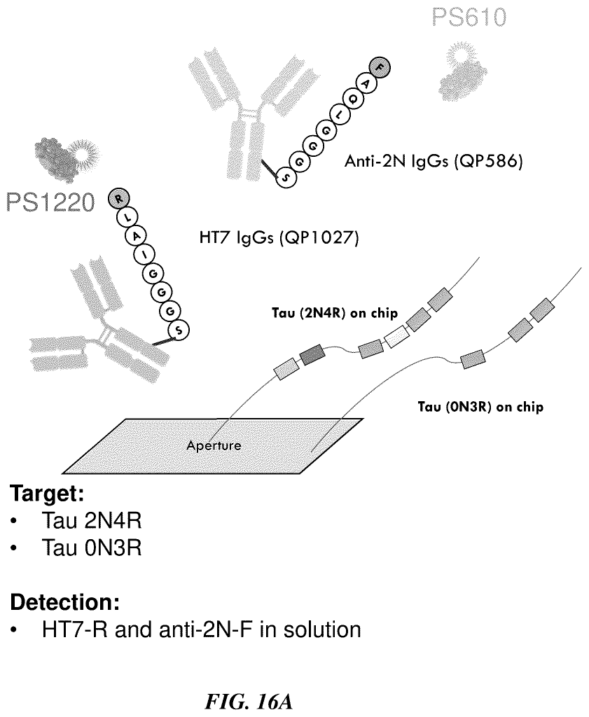

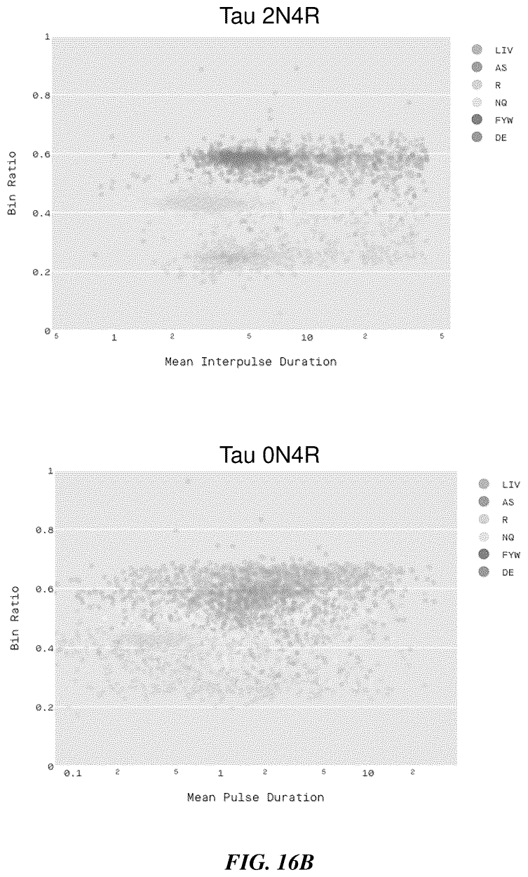

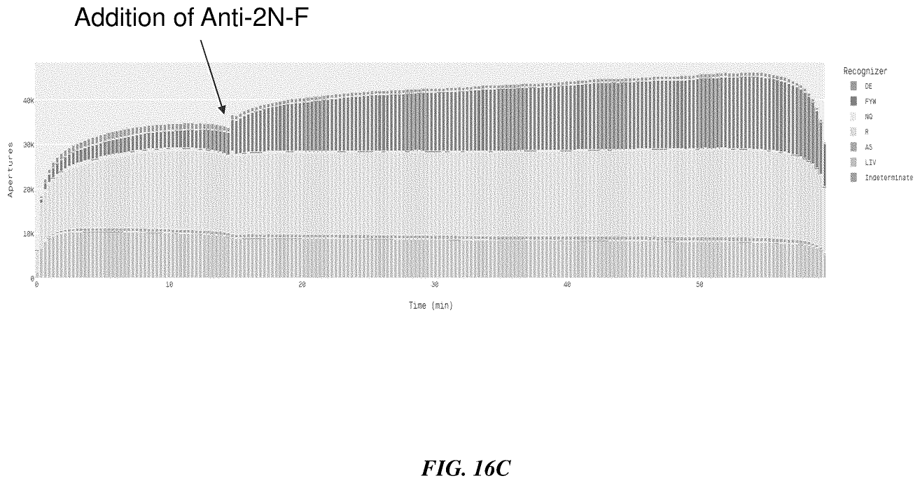

Next, the 2N4R and 0N3R Tau isoforms were immobilized directly on either of the two flow cells of a chip in mixture with the 2N4R-specific antibody, Tau-2N, which carried a peptide label having N-terminal arginine, and the arginine recognizer PS1220 (FIG. 15A). As shown in FIG. 15B, the chip showed pulsing activity in the flow cell carrying 2N4R but not in the flow cell carrying the negative control, indicative of isoform-specific recognition as reported by PS1220. The same 2N4R and 0N3R isoforms were again immobilized on chip and further evaluated, as illustrated in FIG. 16A, using a mixture to permit total and isoform-specific Tau recognition: F-labeled anti-2N IgG (2N4R isoform-specific), R-labeled HT7 (total Tau), PS610 (F recognizer), and PS1220 (R recognizer). As shown by the representative results in FIGS. 16B-16C, total Tau recognition as reported by PS1220 (R recognizer) was observed with both isoforms, whereas isoform-specific recognition as reported by PS610 (F recognizer) was observed only with the surface-immobilized 2N4R.

The ability to capture native Tau or Tau isoforms via capture reagent and without further chemical modification would greatly facilitate clinical samples testing in a diagnostic context. To this end, the Tau antibodies used in the work described above were utilized to implement the sandwich assay for Tau isoform analysis.

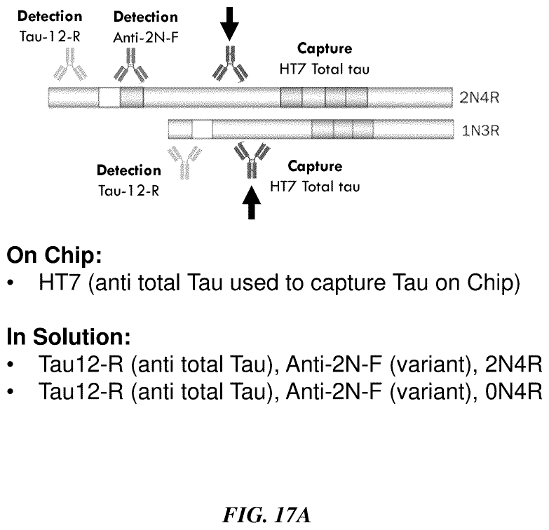

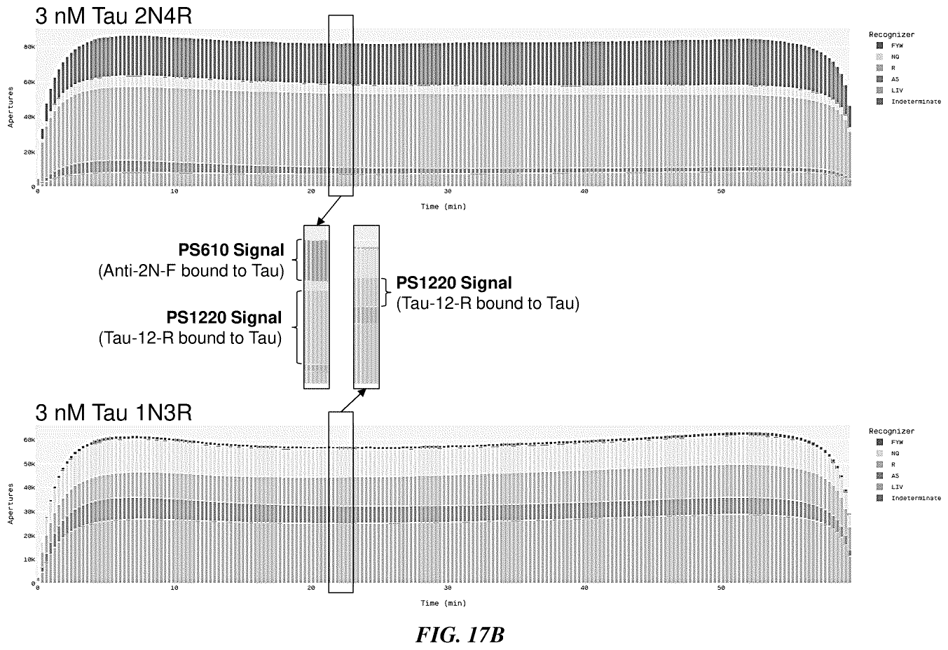

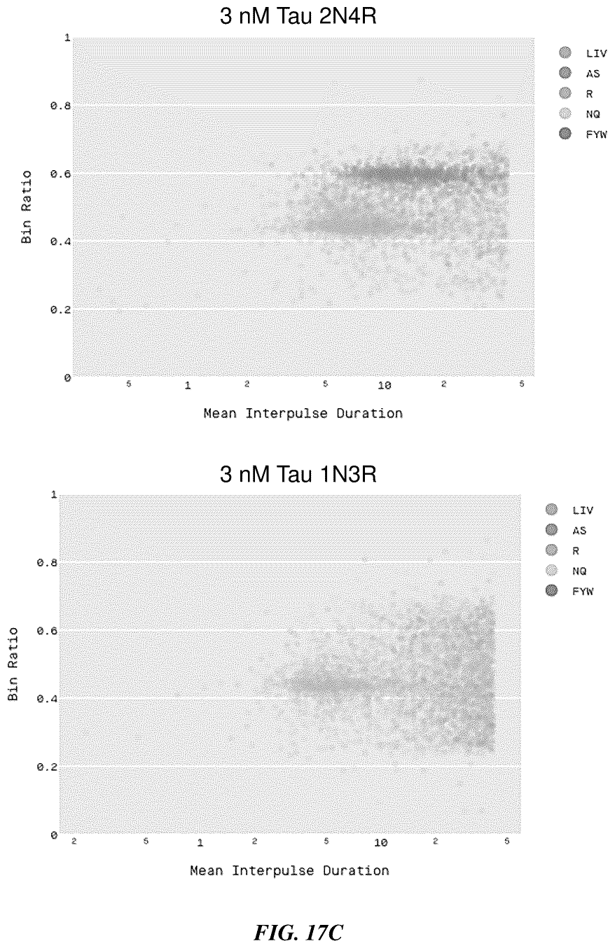

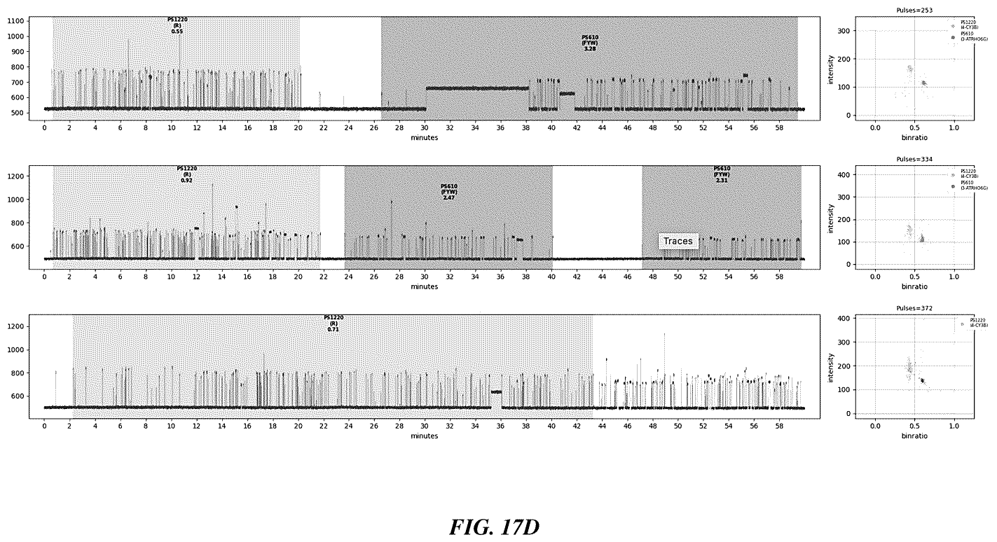

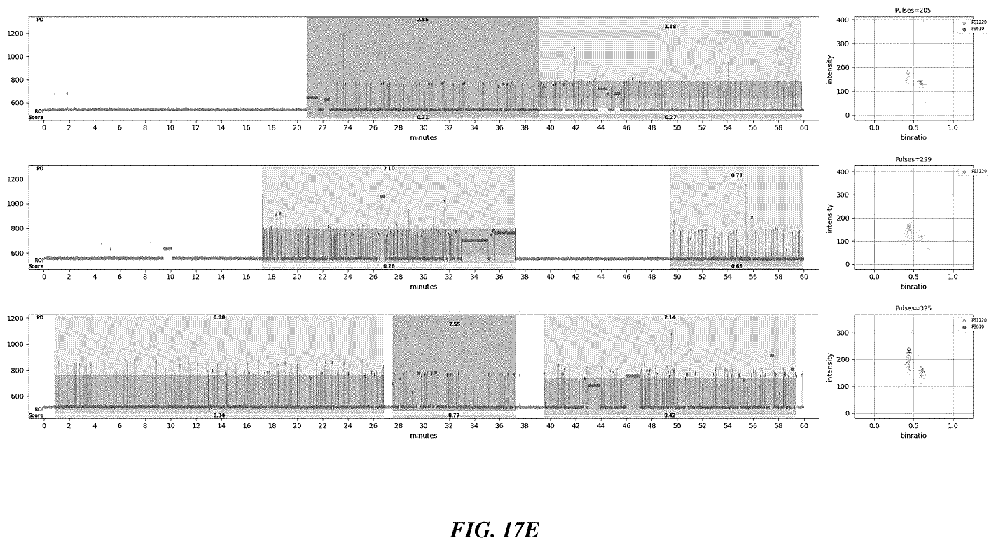

As shown by the scheme in FIG. 17A, total Tau antibody HT7 was immobilized to chip surface to serve as capture reagent for binding either the 2N4R or 1N3R isoform, each of which was evaluated at 3 nM in mixture with Tau-12-R, anti-2N-F, PS1220 (R recognizer), and PS610 (F recognizer). FIG. 17B shows the signal of the binding activity per aperture according to the duration of the recognition experiment with Tau 2N4R analyte (top plot) or Tau 1N3R analyte (bottom plot). Select regions of the plots are shown (middle) to illustrate the detection of total Tau with both isoforms and isoform-specific detection only with the Tau 2N4R analyte. Representative results from these runs are further shown in FIG. 17C, which shows plots of bin ratio as a function of mean interpulse duration. FIGS. 17D-17E each show example signal traces of three apertures in these runs, which demonstrate that colocalization of both antibodies is observed in the same aperture.

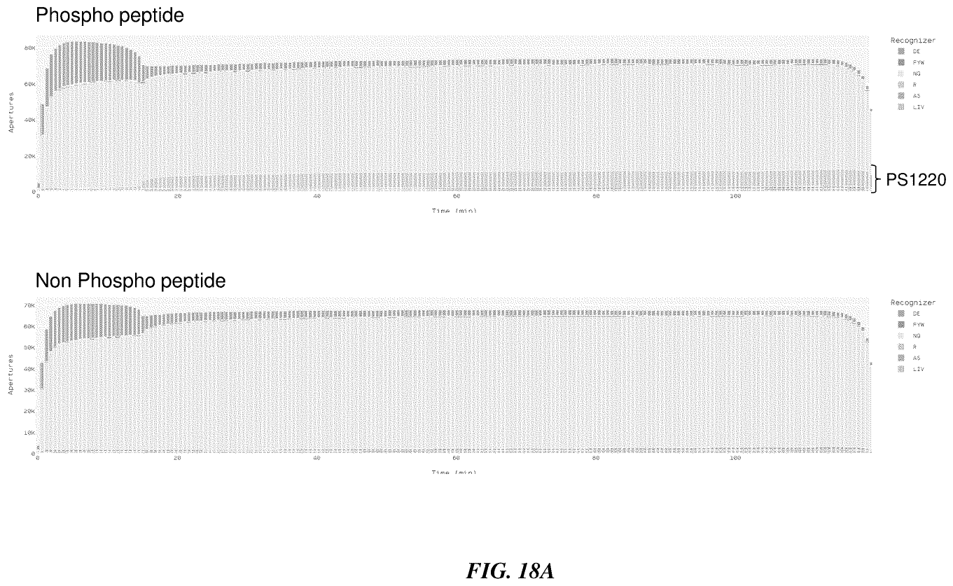

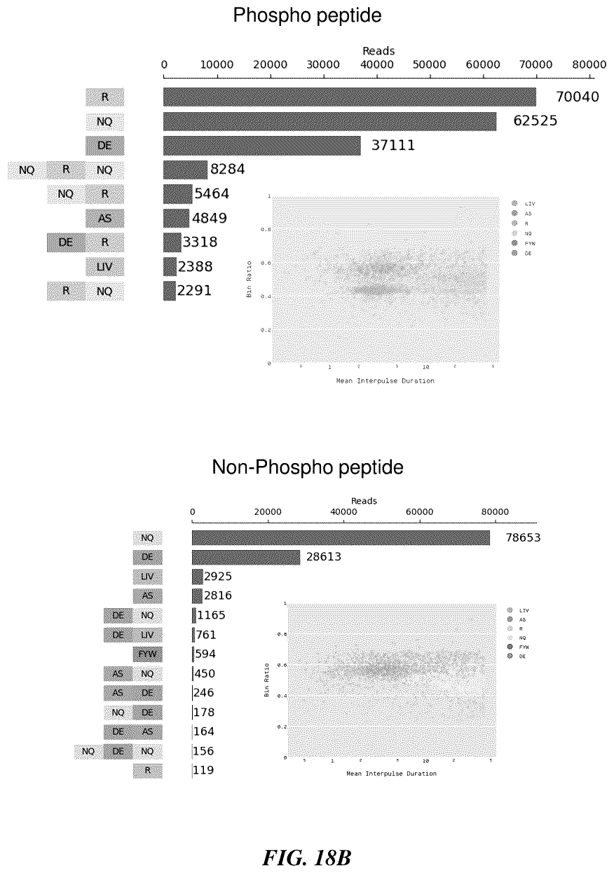

To further evaluate the use of Tau antibodies for isoform-specific detection based on post-translational modification, the AT8 antibody (FIG. 13A) was evaluated as a detection affinity reagent against surface-immobilized phosphorylated Tau peptide (Tau 195-212 (pS202, pT205)) and non-phosphorylated Tau peptide (Tau195-212). AT8 was labeled with N-terminal R peptide for recognition by PS1220. Representative results from these runs are shown in FIGS. 18A-18B. As reported by the PS1220 signal, AT8 exhibited high specificity to the phosphorylated Tau peptide having pS202 and pT205 residues.

### Experimental Methods

### Expression of N-Terminally Labeled VHHs

Synthetic gene fragments encoding a VHH with an N-terminal dye-cycling peptide sequence (cither RLFA, FAQR or LARQ) separated by a G3S linker, and a C-terminal FLAG tag were digested with BbsI, and ligated to plasmid, digested with BbsI and XhoI. Plasmids were transformed into SHuffle T7 Express (BL21) E. coli cells and plasmid sequences confirmed by Sanger sequencing. Final plasmids encode recombinant proteins consisting of, from 5′ to 3′, a Halotag, TEV site, SUMO, dye cycling peptide, GFP and 2×FLAG tag. Constructs to immobilise dye-cycling VHHs on instrument were alternately inserted into plasmid digested with BbsI to incorporate a Sortase A recognition motif. For protein expression, bacteria were grown in Terrific broth with 100 μg ampicillin overnight at 37 C. Cells were diluted to OD 0.1-0.2 and grown at 37 C until OD 0.5-0.6. Protein expression was induced by addition of 0.1 mM IPTG for 4 h. Bacteria were pelleted at 1,000 g for 10 minutes and lysed in 2 mL NEB cell lysis buffer for 30 min. Insoluble fractions were removed by centrifugation at 10,000×g. for 10 minutes. Lysates were incubated for 1 h at 25° C. with 100 μL of Halotag magnetic beads prepared as above in HEB buffer. Covalently bound proteins were then washed 3 ×in 1 ml HEB buffer for 5 minutes. Dye-Cycling VHH was eluted by UlpI SUMO protease (Thermo Fisher) for 1 h at 30 C. GFP was quantified on a nanodrop spectrophotometer by absorbance at 280 nm.

Recognition Runs with Biotinylated Loaded Antibodies

This describes the protocol for loading sequencing chips in recognition runs. Sequencing chips with biotinylated surfaces were first loaded with 40 μL of 50 nM Streptavidin (Recombinant Streptavidin from Streptomyces avidinii (Sigma 85878)) in PBS 1× by mixing up and down through each flow cell 10×, followed by incubation at room temperature for 15 minutes. Each side of the chip was then washed 6× with Wash Buffer 1× by mixing up and down through each flow cell 10×, with excess solution removed after each wash. Following the preceding wash step, 40 μL of 3 nM of biotinylated antibody (ThermoFisher, Biolegend) in PBS 1× was loaded by mixing up and down through each flow cell 10×, followed by incubation at room temperature for 15 minutes. Each side of the chip was then washed 6× with Wash Buffer 1× by mixing up and down through each flow cell 10×, with excess solution removed after each wash. The recognition solution was prepared as described in the Sequencing section of the Platinum Sequencing Protocol V3 but without adding nuclease free water and instead adding 100 nM of N-terminal labeled detection VHH. Finally, the recognition solution was adjusted up to 60 μL final, and 30 μL added to each of two Protein LoBind Eppendorf tubes of 1.5 mL for each side of the chip and 3.3 μL of analyte sample added at 10× of the final concentration of interest.

### N-Terminal Amino Acid Recognizers

The amino acid sequences of N-terminal amino acid recognizers used in these experiments are provided in the table below. These include PS1223 for recognition of tags having N-terminal leucine, isoleucine, or valine, PS610 for recognition of tags having N-terminal phenylalanine, tryptophan, or tyrosine, and PS1220 for recognition of tags having N-terminal arginine. Each recognizer was expressed as a single polypeptide having the sequence indicated below and further appended to a C-terminal tag including a biotin ligase recognition sequence. Following biotinylation of biotin ligase recognition sequences, recognizers were labeled through biotin-streptavidin linkage to dye-labeled molecules.

| Recognizer | Amino Acid sequence |
|---|---|
| PS1223 | MPTAASATESAIEDTPAPARPEVDGRTKPK |
| | RQPRYHVVLWDDDDHTYQYVVVMLRSLFGH |
| | PPSRGYRMAKEMDTQGRVIVLTTTREHAEL |
| | KRDQIHAFGRDRLLARSKGSMKASIEAEEG |
| | SAGSAAGSGEFGSAGSAAGSGEFGSAGSAA |
| | GSGEFMPTAASATESAIEDTPAPARPEVDG |
| | RTKPKRQPRYHVVLWDDDDHTYQYVVVMLR |
| | SLFGHPPSRGYRMAKEMDTQGRVIVLTTTR |
| | EHAELKRDQIHAFGRDRLLARSKGSMKASI |
| | EAEE |
| PS610 | MSDSPVDLKPKPKVKPKLERPKLYKVMLLN |
| | DDYTPMSFVTVVLKAVFRMSEDTGRRVMMT |
| | AHRFGSAVVVVCERDIAETKAKEATDLGKE |
| | AGFPLMFTTEPEEGSAGSAAGSGEFMSDSP |
| | VDLKPKPKVKPKLERPKLYKVMLLNDDYTP |
| | MSFVTVVLKAVFRMSEDTGRRVMMTAHRFG |
| | SAVVVVCERDIAETKAKEATDLGKEAGFPL |
| | MFTTEPEEG |
| PS1220 | MHSKFSHAGRICGAKFKVGEPIYRCKECSF |
| | DDTCVLCVNCFNPKDHLGHHVYTTICTEFN |
| | NGECDCGDKTAWNHTLFCKAEEGSAGSAAG |
| | SGEFMHSKFSHAGRICGAKFKVGEPIYRCK |
| | ECSFDDTCVLCVNCFNPKDHLGHHVYTTIC |
| | TEFNNGECDCGDKTAWNHTLFCKAEEG |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A method of sample analysis, the method comprising:
contacting a capture reagent with a sample comprising one or more analytes, wherein the capture reagent binds an analyte of the sample to form a first complex;
contacting the first complex with a composition comprising one or more affinity reagents and one or more secondary reporters, wherein at least one affinity reagent binds the analyte of the first complex;
detecting at least one series of signal pulses, wherein each series of signal pulses is indicative of a series of binding events between the one or more secondary reporters and an affinity reagent bound to the analyte of the first complex; and
determining a concentration of the analyte in the sample based at least in part on a count of detected series of signal pulses.

2. The method of claim 1, wherein the at least one series of signal pulses comprises:
a first set of at least one series of signal pulses indicative of a first series of binding events between one or more secondary reporters and a first affinity reagent bound to the analyte, and
a second set of at least one series of signal pulses indicative of a second series of binding events between one or more secondary reporters and a second affinity reagent bound to the analyte.

3. The method of claim 2, wherein the first affinity reagent is different from the second affinity reagent.

4. The method of claim 2, wherein the first affinity reagent binds to a first site on the analyte, and wherein the second affinity reagent binds to a second site on the analyte.

5. The method of claim 4, wherein the first site does not comprise a post-translational modification (PTM), and wherein the second site comprises a PTM.

6. The method of claim 4, wherein the first site comprises a first PTM, and wherein the second site comprises a second PTM.

7. The method of any one of claims 1-6, wherein the capture reagent is attached to a surface of a substrate.

8. The method of any one of claims 1-7, further comprising, prior to contacting the capture reagent with the sample:
contacting a substrate with the capture reagent, wherein a surface of the substrate comprises an attachment moiety that forms a covalent or non-covalent attachment between the capture reagent and the surface.

9. The method of any one of claims 1-8, wherein the capture moiety is attached within a first compartment of an array comprising a plurality of compartments.

10. The method of claim 9, wherein the concentration of the analyte in the sample is determined based at least in part on the count of the detected series of signal pulses of the first compartment.

11. The method of claim 9 or 10, wherein the method comprises detecting at least one series of signal pulses in each of at least two compartments of the array.

12. The method of claim 11, wherein the concentration of the analyte in the sample is determined based at least in part on the count of the detected series of signal pulses of the at least two compartments.

13. The method of any one of claims 9-12, wherein a second compartment of the array comprises a second capture moiety bound to a second analyte that is different from the analyte bound by the capture moiety in the first compartment.

14. The method of claim 13, further comprising:
detecting at least one series of signal pulses, wherein each series of signal pulses is indicative of a series of binding events between the one or more secondary reporters and an affinity reagent bound to the second analyte in the second compartment.

15. The method of claim 14, further comprising:
determining a concentration of the second analyte in the sample based at least in part on a count of detected series of signal pulses of the second compartment.

16. The method of claim 14 or 15, further comprising:
determining a concentration of the first and second analytes in the sample based on one or more characteristics of the detected series of signal pulses of the first and second compartments,
optionally wherein the concentration is: a relative concentration of the first analyte to the second analyte, or an absolute concentration of each of the first and second analytes.

17. The method of claim 16, wherein the relative concentration is determined based at least in part on a ratio of the count of detected series of signal pulses of the first compartment to the count of detected series of signal pulses of the second compartment.

18. The method of claim 13, further comprising:
determining a relative concentration of the first and second analytes in the sample based at least in part on a ratio of a total count of detected series of signal pulses across two or more compartments each comprising the first analyte relative to a total count of detected series of signal pulses across two or more compartments each comprising the second analyte.

19. The method of any one of claims 14-18, wherein at least one series of signal pulses detected in each of the first and second compartments comprises a series of signal pulses indicative of analyte binding by an affinity reagent of the same type.

20. The method of claim 19, wherein the series of signal pulses detected in each of the first and second compartments indicate that the analytes in the first and second chambers are encoded by a single gene.

21. The method of claim 20, wherein at least one series of signal pulses detected in the first compartment comprises a series of signal pulses indicative of analyte binding by an affinity reagent of a different type from the at least one series of signal pulses detected in the second compartment.

22. The method of claim 21, wherein the series of signal pulses detected in each of the first and second compartments indicate that the analytes in the first and second chambers are different isoforms encoded by a single gene.

23. The method of any one of claims 1-22, further comprising distinguishing signal pulses indicative of the series of binding events from signal pulses resulting from noise based at least in part on a characteristic pattern in a detected series of signal pulses.

24. The method of claim 23, further comprising removing the signal pulses resulting from noise prior to determining the concentration of the analyte in the sample.

25. The method of claim 23 or 24, wherein the characteristic pattern comprises a pulse duration and/or an interpulse duration of the detected series of signal pulses.

26. The method of claim 25, wherein the pulse duration comprises an average duration of pulses of the detected series of signal pulses.

27. The method of claim 25 or 26, wherein the interpulse duration comprises an average duration between pulses of the detected series of signal pulses.

28. The method of any one of claims 1-27, wherein the capture reagent is contacted with a single composition comprising the sample, the one or more affinity reagents, and the one or more secondary reporters.

29. The method of any one of claims 1-28, wherein the capture reagent is conjugated to a barcode, optionally wherein the barcode is a peptide barcode.

30. The method of claim 29, further comprising:
removing the barcode from the capture reagent; and
determining a sequence of the barcode, wherein the sequence of the barcode is indicative of the analyte to which the capture reagent binds.

31. The method of claim 29 or 30, wherein the capture moiety is attached to a surface through a linkage group comprising the barcode.

32. The method of any one of claims 1-31, wherein the at least one affinity reagent binds the analyte of the first complex to form a second complex comprising the analyte, the capture reagent, and an affinity reagent.

33. The method of claim 32, wherein the capture reagent of the second complex is attached to a surface through a first linkage group, and wherein the affinity reagent is attached to the surface through a second linkage group.

34. The method of claim 33, wherein the capture reagent comprises a first oligonucleotide, and wherein the first oligonucleotide is hybridized to a first surface-immobilized oligonucleotide to form the first linkage group.

35. The method of claim 33 or 34, wherein the affinity reagent comprises a second oligonucleotide, and wherein the second oligonucleotide is hybridized to a second surface-immobilized oligonucleotide to form the second linkage group.

36. The method of any one of claims 33-35, further comprising, prior to detecting the at least one series of signal pulses:
forming the second complex; and
contacting the second complex with the surface to form the first and second linkage groups.

37. A method of sample analysis, the method comprising:
contacting a capture reagent with a sample comprising one or more analytes, wherein the capture reagent binds an analyte of the sample to form a first complex;
contacting the first complex with one or more affinity reagents;
detecting at least one series of signal pulses, wherein each series of signal pulses is indicative of a series of binding events between the one or more affinity reagents and the analyte; and
determining a concentration of the analyte in the sample based at least in part on a count of detected series of signal pulses.

38. The method of claim 37, wherein the at least one series of signal pulses comprises:
a first set of at least one series of signal pulses indicative of a first series of binding events between a first affinity reagent and the analyte, and
a second set of at least one series of signal pulses indicative of a second series of binding events between a second affinity reagent and the analyte.

39. The method of claim 38, wherein the first affinity reagent is different from the second affinity reagent.

40. The method of claim 38, wherein the first affinity reagent binds to a first site on the analyte, and wherein the second affinity reagent binds to a second site on the analyte.

41. The method of claim 40, wherein the first site does not comprise a post-translational modification (PTM), and wherein the second site comprises a PTM.

42. The method of claim 40, wherein the first site comprises a first PTM, and wherein the second site comprises a second PTM.

43. The method of any one of claims 37-42, wherein the capture reagent is attached to a surface of a substrate.

44. The method of any one of claims 37-43, further comprising, prior to contacting the capture reagent with the sample:
contacting a substrate with the capture reagent, wherein a surface of the substrate comprises an attachment moiety that forms a covalent or non-covalent attachment between the capture reagent and the surface.

45. The method of any one of claims 37-44, wherein the capture moiety is attached within a first compartment of an array comprising a plurality of compartments.

46. The method of claim 45, wherein the concentration of the analyte in the sample is determined based at least in part on the count of the detected series of signal pulses of the first compartment.

47. The method of claim 45 or 46, wherein the method comprises detecting at least one series of signal pulses in each of at least two compartments of the array.

48. The method of claim 47, wherein the concentration of the analyte in the sample is determined based at least in part on the count of the detected series of signal pulses of the at least two compartments.

49. The method of any one of claims 45-48, wherein a second compartment of the array comprises a second capture moiety bound to a second analyte that is different from the analyte bound by the capture moiety in the first compartment.

50. The method of claim 49, further comprising:
detecting at least one series of signal pulses, wherein each series of signal pulses is indicative of a series of binding events between the one or more affinity reagents and the second analyte in the second compartment.

51. The method of claim 50, further comprising:
determining a concentration of the second analyte in the sample based at least in part on a count of detected series of signal pulses of the second compartment.

52. The method of claim 50 or 51, further comprising:
determining a concentration of the first and second analytes in the sample based on one or more characteristics of the detected series of signal pulses of the first and second compartments,
optionally wherein the concentration is: a relative concentration of the first analyte to the second analyte, or an absolute concentration of each of the first and second analytes.

53. The method of claim 52, wherein the relative concentration is determined based at least in part on a ratio of the count of detected series of signal pulses of the first compartment to the count of detected series of signal pulses of the second compartment.

54. The method of claim 49, further comprising:
determining a relative concentration of the first and second analytes in the sample based at least in part on a ratio of a total count of detected series of signal pulses across two or more compartments each comprising the first analyte relative to a total count of detected series of signal pulses across two or more compartments each comprising the second analyte.

55. The method of any one of claims 50-54, wherein at least one series of signal pulses detected in each of the first and second compartments comprises a series of signal pulses indicative of analyte binding by an affinity reagent of the same type.

56. The method of claim 55, wherein the series of signal pulses detected in each of the first and second compartments indicate that the analytes in the first and second chambers are encoded by a single gene.

57. The method of claim 56, wherein at least one series of signal pulses detected in the first compartment comprises a series of signal pulses indicative of analyte binding by an affinity reagent of a different type from the at least one series of signal pulses detected in the second compartment.

58. The method of claim 57, wherein the series of signal pulses detected in each of the first and second compartments indicate that the analytes in the first and second chambers are different isoforms encoded by a single gene.

59. The method of any one of claims 37-58, further comprising distinguishing signal pulses indicative of the series of binding events from signal pulses resulting from noise based at least in part on a characteristic pattern in a detected series of signal pulses.

60. The method of claim 59, further comprising removing the signal pulses resulting from noise prior to determining the concentration of the analyte in the sample.

61. The method of claim 59 or 60, wherein the characteristic pattern comprises a pulse duration and/or an interpulse duration of the detected series of signal pulses.

62. The method of claim 61, wherein the pulse duration comprises an average duration of pulses of the detected series of signal pulses.

63. The method of claim 61 or 62, wherein the interpulse duration comprises an average duration between pulses of the detected series of signal pulses.

64. The method of any one of claims 37-63, wherein the capture reagent is contacted with a single composition comprising the sample and the one or more affinity reagents.

65. The method of any one of claims 37-64, wherein the capture reagent is conjugated to a barcode, optionally wherein the barcode is a peptide barcode.

66. The method of claim 65, further comprising:
removing the barcode from the capture reagent; and
determining a sequence of the barcode, wherein the sequence of the barcode is indicative of the analyte to which the capture reagent binds.

67. The method of claim 65 or 66, wherein the capture moiety is attached to a surface through a linkage group comprising the barcode.

68. The method of any one of claims 37-67, wherein at least one affinity reagent binds the analyte of the first complex to form a second complex comprising the analyte, the capture reagent, and an affinity reagent.

69. The method of claim 68, wherein the capture reagent of the second complex is attached to a surface through a first linkage group, and wherein the affinity reagent is attached to the surface through a second linkage group.

70. The method of claim 69, wherein the capture reagent comprises a first oligonucleotide, and wherein the first oligonucleotide is hybridized to a first surface-immobilized oligonucleotide to form the first linkage group.

71. The method of claim 69 or 70, wherein the affinity reagent comprises a second oligonucleotide, and wherein the second oligonucleotide is hybridized to a second surface-immobilized oligonucleotide to form the second linkage group.

72. The method of any one of claims 69-71, further comprising, prior to detecting the at least one series of signal pulses:
forming the second complex; and
contacting the second complex with the surface to form the first and second linkage groups.

73. The method of any one of claims 37-72, wherein the first complex is contacted with a composition comprising the one or more affinity reagents and one or more secondary reporters.

74. The method of claim 73, further comprising:
detecting at least one series of signal pulses indicative of a series of binding events between the one or more secondary reporters and an affinity reagent bound to the analyte of the first complex.

75. A method of determining one or more chemical characteristics of a polypeptide, the method comprising:
contacting a polypeptide with a composition comprising one or more affinity reagents and one or more secondary reporters, wherein at least one affinity reagent binds the polypeptide;
detecting at least one series of signal pulses indicative of a series of binding events between the one or more secondary reporters and an affinity reagent bound to the polypeptide; and
determining one or more chemical characteristics of the polypeptide based on one or more characteristics of the at least one series of signal pulses.

76. The method of claim 75, wherein each series of signal pulses is indicative of a single binding event between an affinity reagent and the polypeptide.

77. The method of claim 75 or 76, wherein each series of signal pulses is indicative of a duration in which the polypeptide is bound by affinity reagent.

78. The method of any one of claims 75-77, wherein each series of signal pulses is separated from another by a duration in which the polypeptide is unbound by affinity reagent.

79. The method of any one of claims 75-78, wherein the polypeptide is attached to a surface.

80. The method of claim 79, wherein the polypeptide is attached to the surface through a capture reagent that binds the polypeptide.

81. The method of claim 80, wherein the capture reagent binds to a site on the polypeptide that is different from a site to which the at least one affinity reagent binds.

82. The method of any one of claims 75-81, further comprising:
contacting the polypeptide with a capture reagent that binds the polypeptide, wherein the capture reagent is attached to a surface.

83. The method of claim 82, wherein the polypeptide is contacted with the capture reagent prior to contacting the polypeptide with the composition.

84. The method of claim 82, wherein the polypeptide is contacted with the capture reagent in a single composition comprising the polypeptide, the one or more affinity reagents, and the one or more secondary reporters.

85. The method of any one of claims 82-84, further comprising, prior to contacting the polypeptide with the capture reagent:
contacting the capture reagent with the surface, wherein the surface comprises an attachment moiety that forms a covalent or non-covalent attachment between the capture reagent and the surface.

86. The method of claim 85, wherein the attachment moiety comprises an avidin protein, and wherein the capture reagent comprises a biotin moiety that is bound by the avidin protein.

87. The method of any one of claims 80-86, wherein the capture reagent comprises an antibody, an antigen-binding portion of an antibody (e.g., a single-chain antibody variable fragment (scFv) or V_{H}H fragment), or an aptamer.

88. The method of any one of claims 75-87, wherein contacting the polypeptide with the composition comprises:
contacting the polypeptide with a single composition comprising the one or more affinity reagents and the one or more secondary reporters.

89. The method of any one of claims 75-87, wherein contacting the polypeptide with the composition comprises:
contacting the polypeptide with a first composition comprising the one or more affinity reagents and a second composition comprising the one or more secondary reporters.

90. The method of any one of claims 75-89, wherein the one or more secondary reporters bind the affinity reagent at a faster rate than a time required for the affinity reagent to dissociate from the polypeptide.

91. The method of any one of claims 75-90, wherein the one or more affinity reagents comprise one or more antibodies, antigen-binding portions of an antibody (e.g., a single-chain antibody variable fragment (scFv) or V_{H}H fragment), or aptamers.

92. The method of any one of claims 75-91, wherein the one or more secondary reporters comprise one or more terminal amino acid recognizers.

93. The method of any one of claims 75-92, wherein each of the one or more affinity reagents is conjugated to a tag peptide.

94. The method of claim 93, wherein each of the one or more secondary reporters binds the tag peptide of an affinity reagent.

95. The method of claim 93 or 94, wherein each series of signal pulses is indicative of a series of binding events between the one or more secondary reporters and the tag peptide of an affinity reagent bound to the polypeptide.

96. The method of any one of claims 93-95, wherein each of the one or more secondary reporters comprises a terminal amino acid recognizer that binds a terminal amino acid of the tag peptide.

97. The method of any one of claims 75-92, wherein each of the one or more affinity reagents is conjugated to a tag oligonucleotide.

98. The method of claim 97, wherein each of the one or more secondary reporters comprises a complementary oligonucleotide that hybridizes to the tag oligonucleotide of an affinity reagent.

99. The method of claim 97 or 98, wherein each series of signal pulses is indicative of a series of hybridization events between the one or more secondary reporters and the tag oligonucleotide of an affinity reagent bound to the polypeptide.

100. The method of any one of claims 75-99, wherein each of the one or more secondary reporters comprises a luminescent label.

101. The method of any one of claims 75-100, wherein the polypeptide is a full-length protein or a polypeptide fragment thereof.

102. The method of any one of claims 75-101, wherein the one or more affinity reagents comprise at least two affinity reagents that bind different proteoforms of the polypeptide.

103. The method of any one of claims 75-102, wherein the one or more affinity reagents comprise at least two affinity reagents that bind different epitopes of a single proteoform of the polypeptide.

104. The method of any one of claims 75-103, wherein the one or more affinity reagents comprise a first affinity reagent that binds a first epitope of the polypeptide and a second affinity reagent that binds a second epitope of the polypeptide.

105. The method of claim 104, wherein the first epitope does not comprise a post-translational modification (PTM), and wherein the second epitope comprises a PTM.

106. The method of claim 104 or 105, wherein the first affinity reagent is a protein-specific affinity reagent that binds different proteoforms of the polypeptide, and wherein the second affinity reagent is a PTM-specific affinity reagent that binds a specific proteoform of the polypeptide.

107. The method of any one of claims 75-106, wherein the one or more characteristics of the at least one series of signal pulses comprise a first recognition segment duration of a first series of signal pulses.

108. The method of claim 107, wherein the first recognition segment duration comprises a length of time during which the first series of signal pulses is detected.

109. The method of claim 107 or 108, wherein the first recognition segment duration is characteristic of a dissociation rate of affinity reagent binding and/or a dissociation rate of capture reagent binding.

110. The method of any one of claims 107-109, wherein the one or more characteristics of the at least one series of signal pulses comprise an average of two or more recognition segment durations.

111. The method of any one of claims 75-110, wherein the one or more characteristics of the at least one series of signal pulses comprise an intersegment duration between two recognition segment durations.

112. The method of claim 111, wherein the intersegment duration comprises a length of time between two successively detected series of signal pulses.

113. The method of claim 111 or 112, wherein the intersegment duration is characteristic of an association rate of affinity reagent binding and/or an association rate of capture reagent binding.

114. The method of any one of claims 111-113, wherein the one or more characteristics of the at least one series of signal pulses comprise an average of two or more intersegment durations.

115. The method of any one of claims 75-114, wherein the one or more characteristics of the at least one series of signal pulses comprise a first pulse duration of a first series of signal pulses.

116. The method of claim 115, wherein the first pulse duration comprises an average duration of pulses of the first series of signal pulses.

117. The method of claim 115 or 116, wherein the first pulse duration is characteristic of a dissociation rate of secondary reporter binding.

118. The method of any one of claims 75-117, wherein the one or more characteristics of the at least one series of signal pulses comprise a first interpulse duration of a first series of signal pulses.

119. The method of claim 118, wherein the first interpulse duration comprises an average duration between pulses of the first series of signal pulses.

120. The method of claim 118 or 119, wherein the first interpulse duration is characteristic of an association rate of secondary reporter binding.

121. The method of any one of claims 75-120, wherein determining the one or more chemical characteristics of the polypeptide comprises identifying the polypeptide.

122. The method of any one of claims 75-121, wherein determining the one or more chemical characteristics of the polypeptide comprises identifying one or more post-translational modifications of the polypeptide.

123. The method of any one of claims 75-122, wherein determining the one or more chemical characteristics of the polypeptide comprises determining a concentration of the polypeptide in a sample from which it was derived.

124. A single-molecule method comprising:
(a) contacting a single polypeptide with one or more post-translational modification-specific (PTM-specific) affinity reagent to produce one or more polypeptide-affinity reagent complexes, optionally wherein each affinity reagent is an antibody that binds to the single polypeptide;
(b) contacting the polypeptide-affinity reagent complexes with one or more luminescently labeled secondary reporters, wherein each of the secondary reporters specifically binds to an affinity reagent; and
(c) identifying whether the single polypeptide comprises a post-translational modification (PTM) by determining a luminescence signature representative of the binding interaction(s) between the polypeptide-affinity reagent complex and the one or more PTM-specific affinity reagents.

125. The method of claim 124, wherein the method further comprises:
(d) contacting the single polypeptide with one or more terminal amino acid recognition molecules; and
(e) detecting a series of signal pulses indicative of association of the one or more terminal amino acid recognition molecules with successive amino acids exposed at a terminus of the single polypeptide while the single polypeptide is being degraded.

126. A method of polypeptide sequencing comprising:
(a) contacting a chip array comprising a plurality of compartments with a plurality of polypeptides;
(b) immobilizing each polypeptide of the plurality of polypeptides to a surface of the chip array;
(c) contacting each polypeptide of the plurality of polypeptides with one or more affinity reagents to produce a plurality of polypeptide-affinity reagent complexes, optionally wherein each affinity reagent is an antibody that binds to one of the single polypeptides;
(d) contacting the polypeptide-affinity reagent complexes with one or more luminescently labeled secondary reporters, wherein each of the secondary reporters specifically binds to an affinity reagent; and
(e) determining the luminescence signature representative of the binding interaction(s) between each polypeptide-affinity reagent complex and the one or more affinity reagents.

127. The method of claim 126, wherein the method further comprises:
(f) contacting each polypeptide with one or more terminal amino acid recognition molecules; and
(g) detecting a series of signal pulses indicative of association of the one or more terminal amino acid recognition molecules with successive amino acids exposed at a terminus of each polypeptide while each polypeptide is being degraded, thereby sequencing each polypeptide.

128. A method of characterizing proteoforms of a polypeptide comprising:
(a) contacting a chip array comprising a plurality of compartments with a sample comprising a first proteoform of a polypeptide and a second proteoform of a polypeptide, wherein the post-translational modification (PTM) profile of the first proteoform is different than the PTM profile of the second proteoform;
(b) immobilizing the first proteoform to a surface of a first compartment of the chip array and the second proteoform to a surface of a second compartment of the chip array;
(c) contacting the first proteoform and the second proteoform with one or more affinity reagents to produce a plurality of first polypeptide-affinity reagent complexes and a plurality of second polypeptide-affinity reagent complexes, optionally wherein each affinity reagent is an antibody that binds to one of the single polypeptides;
(d) contacting the polypeptide-affinity reagent complexes with one or more luminescently labeled secondary reporters, wherein each of the secondary reporters specifically binds to an affinity reagent; and
(e) identifying the first proteoform and/or the second proteoform by determining the luminescence signature representative of the binding interaction(s) between each proteoform and the one or more affinity reagents.

129. The method of claim 128, wherein the method further comprises:
(f) contacting the first proteoform and/or the second proteoform with one or more terminal amino acid recognition molecules; and
(g) detecting a series of signal pulses indicative of association of the one or more terminal amino acid recognition molecules with successive amino acids exposed at a terminus of each proteoform while each proteoform is being degraded, thereby sequencing the first proteoform and the second proteoform.

130. The method of any one of claims 126 to 129, wherein the one or more affinity reagents comprise one or more PTM-specific affinity reagents.

131. The method of any one of claims 124-130, wherein the one or more PTM-specific affinity reagents are antibodies or aptamers.

132. The method of any one of claims 124-131, wherein the one or more PTM-specific affinity reagents specifically bind to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination.

133. The method of any one of claims 124-132, wherein the one or more PTM-specific affinity reagents specifically bind to phospho-tyrosine, phospho-serine, or phospho-threonine.

134. The method of any one of claims 124-133, wherein the one or more PTM-specific affinity reagents is labeled.

135. The method of claim 134, wherein the label is a luminescent label or a conductivity label.

136. The method of claim 135, wherein the luminescent label comprises at least one fluorophore dye molecule.

137. The method of claim 136, wherein the luminescent label comprises 20 or fewer fluorophore dye molecules.

138. The method of any one of claims 124-137, wherein the polypeptide(s) are contacted with two or more PTM-specific affinity reagents at the same time.

139. The method of claim 138, wherein each of the two or more PTM-specific affinity reagents comprise a unique label relative to the other PTM-specific affinity reagents.

140. The method of any one of claims 124-139, wherein the polypeptide(s) are contacted in series with a first PTM-specific affinity reagent and a second PTM-specific affinity reagent, optionally wherein the first PTM-specific affinity reagent is removed (e.g., by washing) prior to addition of the second PTM-specific affinity reagent.

141. The method of any one of claims 124-140, wherein determining the luminescence signature comprises detecting a series of signal pulses indicative of association of the one or more terminal PTM-specific affinity reagents with the PTM of the polypeptide(s).

142. The method of claim 141, wherein detecting a series of signal pulses indicative of association of the one or more terminal PTM-specific affinity reagents with the PTM of the polypeptide(s) allows for a determination of the type of amino acids located at positions in proximity to the PTM of the polypeptide(s).

143. The method of claim 141, wherein detecting a series of signal pulses indicative of association of the one or more terminal PTM-specific affinity reagents with the PTM of the polypeptide(s) allows for a determination of the location of the PTM within the polypeptide(s).

144. The method of claim 141, wherein detecting a series of signal pulses indicative of association of the one or more terminal PTM-specific affinity reagents with the PTM of the polypeptide(s) assists with a determination of the amino acid sequence of the polypeptide(s).

145. The method of any one of claims 124-144, wherein the PTM is to an amino acid comprising a phosphorylation, a glycosylation, acetylation, ADP-ribosylation, citrullination, formylation, N-linked glycosylation, O-linked glycosylation, hydroxylation, methylation, myristoylation, neddylation, nitration, oxidation, palmitoylation, prenylation, S-nitrosylation, sulfation, sumoylation, or ubiquitination.

146. The method of any one of claims 124-145, wherein the PTM is phospho-tyrosine, phospho-serine, or phospho-threonine.

147. The method of any one of claims 124-146, wherein contacting the polypeptide(s) with one or more terminal amino acid recognition molecules further comprises contacting the polypeptide(s) with a cleaving reagent.

148. The method of claim 147, wherein the cleaving reagent is an aminopeptidase.

149. The method of any one of claims 124-148, wherein the method allows for identification of the presence of the PTM at any location in the polypeptide(s).

150. The method of any one of claims 124-149, further comprising washing the polypeptide(s) after determining the luminescence signature of the polypeptide(s) in the presence of the one or more PTM-specific affinity reagents.

151. The method of any one of claims 124-150, further comprising fragmenting the polypeptide(s) prior to step (a).

152. The method of claim 151, wherein fragmenting by cleaving (e.g., chemically cleaving) and/or digesting (e.g., enzymatically digesting using a peptidase) the polypeptide(s).

153. The method of any one of claims 124-152, wherein association of the one or more terminal amino acid recognition molecules with each type of amino acid exposed at the terminus produces a characteristic pattern in the series of signal pulses that is different from other types of amino acids exposed at the terminus, optionally wherein the characteristic pattern comprises a portion of the series of signal pulses.

154. The method of claim 153, wherein a signal pulse of the characteristic pattern corresponds to an individual association event between a terminal amino acid recognition molecule and an amino acid exposed at the terminus.

155. The method of claim 154, wherein the signal pulse of the characteristic pattern comprises a pulse duration that is characteristic of a dissociation rate of binding between the terminal amino acid recognition molecule and the amino acid exposed at the terminus.

156. The method of claim 155, wherein each signal pulse of the characteristic pattern is separated from another by an interpulse duration that is characteristic of an association rate of terminal amino acid recognition molecule binding.

157. The method of any one of claims 153-156, wherein the characteristic pattern corresponds to a series of reversible terminal amino acid recognition molecule binding interactions with the amino acid exposed at the terminus of the single polypeptide molecule.

158. The method of any one of claims 153-157, wherein the characteristic pattern is indicative of the amino acid exposed at the terminus of the single polypeptide molecule and an amino acid at a contiguous position.

159. A method of screening for modulators of a target protein comprising:
(a) contacting a chip array comprising a plurality of compartments with a library of different compounds;
(b) immobilizing each of the different compounds of the library to a surface of the chip array;
(c) contacting the library of different compounds with a target protein; and
(d) determining the luminescence signature representative of the binding interaction(s) between at least one different compound and the target protein, thereby identifying whether the at least one different compound is a modulator of the target protein.

160. The method of claim 159, wherein the library of different compounds comprises at least two, at least five, at least 10, at least 20, at least 30, at least 50, at least 75, at least 100, at least 200, at least 250, at least 500, or at least 1000 different compounds.

161. The method of claim 159 or 160, wherein each of the different compounds is independently a small molecule or a peptide, optionally wherein the peptide is 50-500 Daltons in size.

162. The method of any one of claims 159-161, wherein the target protein is labeled with a luminescent label or a conductivity label.

163. The method of claim 162, wherein the luminescent label comprises at least one fluorophore dye molecule and/or the luminescent label comprises 20 or fewer fluorophore dye molecules.

164. The method of any one of claims 159-163, wherein, following (b), each compartment of the chip array contains a single copy of a different compound.

165. The method of any one of claims 159-164, wherein (d) comprises determining the luminescence signature representative of the binding interaction(s) between at least two, at least five, at least 10, at least 20, at least 30, at least 50, at least 75, at least 100, at least 200, at least 250, at least 500, or at least 1000 of the different compounds and the target protein.

166. The method of any one of claims 159-164, wherein (d) comprises determining the luminescence signature representative of the binding interaction(s) between each of the different compounds and the target protein.

167. The method of any one of claims 159-166, wherein determining a luminescence signature representative of binding interaction(s) comprises:
determining residence time for the interaction between a compound and the target protein; and/or
determining the binding affinity of a compound for the target protein.

168. The method of any one of claims 159-167, wherein determining the luminescence signature representative of the binding interaction(s) between at least one different compounds and the target protein comprises determining whether the at least one different compound is a modulator of the target protein.

169. The method of any one of claims 159-168, wherein (d) comprises identifying whether the at least one different compound is a negative regulator (e.g., inhibitor) or positive regulator of the target protein.

170. The method of any one of claims 159-169, wherein the method further comprises, following (d), a step of contacting the different compounds with one or more post-translational modification specific (PTM-specific) affinity reagents; and identifying whether each compound comprises a post-translational modification (PTM) by determining the luminescence signature representative of the binding interaction(s) between each compound and the one or more PTM-specific affinity reagents.

171. The method of any one of claims 159-170, wherein the method further comprises:
(e) optionally washing away the target protein;
(f) contacting each of the different compounds with one or more terminal amino acid recognition molecules, wherein each of the different compounds is a different peptide; and
(g) detecting a series of signal pulses indicative of association of the one or more terminal amino acid recognition molecules with successive amino acids exposed at a terminus of each different peptide while each peptide is being degraded, thereby sequencing each peptide.

172. The method of any one of claims 159-171, wherein each of the different compounds is immobilized to the chip array using a peptide linker.

173. The method of claim 172, wherein each of the different compounds is a small molecule.

174. The method of any one of claims 159-173, wherein each of the different compounds is linked to a barcode, optionally wherein the barcode is a peptide barcode or a nucleic acid barcode.

175. A method of screening for modulators of a target protein comprising:
(a) contacting a chip array comprising a plurality of compartments with a target protein;
(b) immobilizing the target protein to a surface of the chip array;
(c) contacting the target protein with a library of different compounds; and
(d) determining the luminescence signature representative of the binding interaction(s) between at least one different compound and the target protein, thereby identifying whether the at least one different compound is a modulator of the target protein.

176. The method of claim 175, wherein the library of different compounds comprises at least two, at least five, at least 10, at least 20, at least 30, at least 50, at least 75, at least 100, at least 200, at least 250, at least 500, or at least 1000 different compounds.

177. The method of claim 175 or 176, wherein each of the different compounds is independently a small molecule or a peptide, optionally wherein the peptide is 50-500 Daltons in size.

178. The method of any one of claims 175-177, wherein each of the different compounds is labeled with a luminescent label or a conductivity label.

179. The method of claim 178, wherein the luminescent label comprises at least one fluorophore dye molecule and/or the luminescent label comprises 20 or fewer fluorophore dye molecules.

180. The method of any one of claims 175-179, wherein, following (b), each compartment of the chip array contains a single copy of the target protein.

181. The method of any one of claims 175-180, wherein (d) comprises determining the luminescence signature representative of the binding interaction(s) between at least two, at least five, at least 10, at least 20, at least 30, at least 50, at least 75, at least 100, at least 200, at least 250, at least 500, or at least 1000 of the different compounds and the target protein.

182. The method of any one of claims 175-181, wherein (d) comprises determining the luminescence signature representative of the binding interaction(s) between each of the different compounds and the target protein.

183. The method of any one of claims 175-182, wherein determining a luminescence signature representative of binding interaction(s) comprises:
determining residence time for the interaction between a compound and the target protein; and/or
determining the binding affinity of a compound for the target protein.

184. The method of any one of claims 175-183, wherein determining the luminescence signature representative of the binding interaction(s) between at least one different compounds and the target protein comprises determining whether the at least one different compound is a modulator of the target protein.

185. The method of any one of claims 175-184, wherein (d) comprises identifying whether the at least one different compound is a negative regulator (e.g., inhibitor) or positive regulator of the target protein.

186. The method of any one of claims 175-185, wherein each of the different compounds is linked to a barcode, optionally wherein the barcode is a peptide barcode or a nucleic acid barcode.
